(19) United States
(12) Patent Application Publication
(10) Pub. No.: US 2007/0237763 A1
(43) Pub. Date: Oct. 11, 2007

(54) Compositions and methods for the treatment of cancer

(75) Inventors: Jacques F. Banchereau, Dallas, TX (US); Anna Karolina Palucka, Dallas, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 11/544,229

(22) Filed: Oct. 6, 2006

Related U.S. Application Data
(60) Provisional application No. 60/831,984, filed on Jul. 19, 2006.
(60) Provisional application No. 60/724,316, filed on Oct. 6, 2005.

(51) Int. Cl. A61K 39/395; A61K 45/00

(52) U.S. Cl. 424/133.1; 424/158.1; 424/278.1

(57) ABSTRACT

The present invention includes compositions and methods for the treatment of cancers by controlling the type of immune response mounted against the tumor, and more particularly, the treatment of tumors with cytokine antagonists to change the type of helper T cell response and inhibition of angiogenesis, prevention of formation of metastasis and prevention of formation of tumor stroma.

### CROSS-REFERENCE TO RELATED APPLICATIONS

This application claims priority to U.S. Provisional Application Ser. No. 60/831,984, filed Jul. 19, 2006, and U.S. Provisional Application Serial No. 60,724,316, filed Oct. 6, 2005, the contents of each of which are incorporated by reference herein in their entirety.

### STATEMENT OF FEDERALLY FUNDED RESEARCH

This invention was made with U.S. Government support under Contract No. RO-1 CA89440, R21 AI056001, U19 AIO57234, RO-1 CA78846 and CA85540 awarded by the NIH. The government has certain rights in this invention.

### TECHNICAL FIELD OF THE INVENTION

The present invention relates in general to the field of cancer treatment, and more particularly, to the characterization and development of novel treatment against cancer.

### BACKGROUND OF THE INVENTION

Without limiting the scope of the invention, its background is described in connection with the host response to cancers. Cancer growth and development depends on the interaction between cancer cells and surrounding nonmalignant stroma composed of non-hematopoietic cells (fibroblasts, endothelial cells) and immune cells from both the innate and the adaptive immune system (Coussens and Werb, 2002; Joyce, 2005). Innate immune cells consist of neutrophils, macrophages (MeΦ), dendritic cells (DCs), mast cells, and NK cells (Janeway and Medzhitov, 2002). The adaptive immune cells are T and B lymphocytes capable of immune memory and rapid response upon antigen re-encounter. However, lymphocytes need to be educated as to the nature of the antigen. This task falls upon DCs that bridge the two arms of the immune system (Banchereau et al., 2000; Banchereau and Steinman, 1998; Shortman and Liu, 2002; Steinman, 1991). Indeed, DCs induce and maintain immune response and as opposed to MΦ, are able to prime naïve lymphocytes. Furthermore, vaccination with antigen loaded-DCs in both mouse and humans has shown that DCs can break tolerance to cancer and educate T cells (Banchereau and Palucka, 2005). Therefore, DCs represent an early target for manipulation by tumor.

Many studies have focused on the role of tumor associated macrophages (TAMs) and their subsets (Balkwill et al., 2005; Condeelis and Pollard, 2006). However, less attention has been given to DCs. Many studies in humans observed infiltration of tumors with DC (Gabrilovich, 2004). Yet, the immunological consequences of DC infiltration are less well understood. Tumors are thought to escape immune effectors via subverting DC function (Gabrilovich, 2004). For example, activation of STAT-3 in myeloid cells results in the increased production of vascular endothelial growth factor (VEGF) (Wang et al., 2004) that interferes with DC maturation (Gabrilovich et al., 1996). IL-6 secreted by breast cancer cells skews monocyte differentiation into TAM at the expense of DC (Chomarat et al., 2000) thereby skewing antigen presentation towards antigen degradation (Delamarre et al., 2005). Finally, tumors promote differentiation of IL-10 and/or TGF-β secreting subset of DCs that in turn expands CD4⁺CD25⁺ regulatory T cells (Enk et al., 1997; Ghiringhelli et al., 2005; Levings et al., 2005). However, there is still a need for compositions and methods for improving the type of immune response that is mounted by the host against cancer cells.

### SUMMARY OF THE INVENTION

It has been found that blocking of IL-13 (interleukin-13) can be used to treat tumors of epithelial origin. The present invention includes compositions and methods of modulating a T cell response to cancer by identifying a patient in need of cancer treatment in which the predominate immune response includes the secretion of Type II cytokines; and treating the affected tissue with one or more Type II cytokine antagonists, wherein the Type II cytokine antagonists block CD4+ T cells that secrete Type II cytokines and increase the percentage of Th1 T cells in the affected tissue. It has been found that the present invention may be used for the treatment of cancers of epithelial origin, e.g., prostate and breast. The Type II cytokine antagonists include, e.g., IL-13, but may also include anti-IL-4, IL-5, IL-9, IL-13 or IL-25 antibody, a humanized anti-IL-4, IL-5, IL-9, IL-13 or IL-25 antibody, and combinations thereof. In another embodiment, Type II cytokine antagonists may be inactivated IL-4, IL-5, IL-9, IL-13 or IL-25 and combinations thereof. These antagonists may be provided in a single or multiple doses. Type II cytokines may be prevented using a variety and combinations of active agents, e.g., anti-cytokine receptors, neutralizing antibodies, receptor antagonists, soluble receptors, molecules interfering in the receptor-ligand binding, and inhibitors of downstream events of type II signaling pathways.

Alternatively, the Type II cytokine antagonists may be soluble IL-4, IL-5, IL-9, IL-13 or IL-25 receptors and combinations thereof. In fact, the present invention may use combinations of one or more anti-cytokine antibodies (e.g., humanized antibodies), receptor antagonists and inactivated cytokine. One specific example may be a blocking IL-13 receptor binding antibody, a soluble IL-13R and combinations thereof. Other embodiments include the use of an IL-13 antagonist to decrease CD4+ T cells that secrete Type II cytokines (and increase CD4+ T cells that secrete Type I cytokines).

In certain embodiments it may also be useful to include anti-IFN-γ antibody, a humanized anti-IFN-γ antibody, a soluble IFN-γ receptor and combinations thereof to antagonize the effects of IFN-γ. In yet other embodiment, the antagonists may also include an anti-TNF antibody, a humanized anti-TNF antibody, a soluble TNF receptor and combinations thereof.

The present invention also includes compositions and methods for improving T cell responses to breast cancer by identifying a patient in need of treatment for a breast cancer and treating the affected tissue with one or more IL-13 antagonists, wherein the IL-13 antagonists block CD4+ T cells that secrete Type II cytokines. Examples of IL-13 antagonists include a blocking IL-13 receptor binding antibody, an inactivated IL-13, a soluble IL-13R and combinations thereof.

The composition of the present invention may be used in conjunction with a method to improve immunity against breast cancer by administering to a patient in need thereof a therapeutically effective amount of one or more Type II cytokine antagonists. Examples of Type II cytokine antagonists include, e.g., anti-IL-4, IL-5, IL-9, IL-13 or IL-25 antibody, a humanized anti-IL-4, IL-5, IL-9, IL-13 or IL-25 antibody; inactivated IL-4, IL-5, IL-9, IL-13 or IL-25 and combinations thereof; soluble IL-4, IL-5, IL-9, IL-13 or IL-25 receptors and combinations thereof, and combinations of two or more of the listed antagonists. The composition may also include anti-IFN-γ antibody, a humanized anti-IFN-γ antibody, a soluble IFN-γ receptor and combinations thereof and/or an anti-TNF antibody, a humanized anti-TNF antibody, a soluble TNF receptor and combinations thereof.

In yet another embodiment, the compositions and methods of the present invention may also includes one or more Type I cytokines to stimulate Th1 responses against the cancer. The one or more Type I cytokines may be provided in a single or multiple dose that stimulate Th1 responses.

The present invention also includes a method of reducing Th2 polarization by human breast cancer by providing an effective amount of one or more Type II cytokine antagonists selected from anti-IL-4, IL-5, IL-9, IL-13 or IL-25; soluble receptors for IL-4, IL-5, IL-9, IL-13 or IL-25 and combinations thereof; an anti-IFN-γ antibody, a humanized anti-IFN-γ antibody, a soluble IFN-γ receptor and combinations thereof and/or an anti-TNF antibody, a humanized anti-TNF antibody, a soluble TNF receptor and combinations thereof.

The present invention also includes compositions and methods for inhibiting angiogenesis in tumors in which a subject or patient in need thereof is provided with an effective amount of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof. The method for inhibiting angiogenesis may be used alone or in combination with any of the therapies taught and discussed herein.

Alternatively, the present invention also includes compositions and method for inhibiting the function of tumor associated macrophages by cancers of epithelial cell origin in which an effective amount of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof is provided to a patient in need thereof.

The present invention also includes compositions and methods for prevention of metastasis and/or the formation of tumor stroma by providing an amount effective to prevent the metastasis and/or formation of tumor stroma of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.

### BRIEF DESCRIPTION OF THE DRAWINGS

For a more complete understanding of the features and advantages of the present invention, reference is now made to the detailed description of the invention along with the accompanying figures and in which:

FIGS. 1a and 2a shows the presence of Type 2 cytokines in the microenvironment of breast cancer samples from patients. Cytokine concentration measured by Multiplex Cytokine Bead assay (pg/ml, ordinate) in tumor cell suspensions from breast tumor (black, T) and surrounding tissue (white, ST) (FIG. 1a) or whole tumor fragments (FIG. 1b) after overnight activation with PMA and lonomycin. Mean±SD

FIGS. 2a to 2d show IL-13 secreting CD4⁺T cells in breast cancer samples from patients. Flow cytometry analysis of single cell tumor suspensions. (FIG. 2a) Gating of CD4+T cell infiltrate. FIG. 2b shows two patterns of intracytoplasmic staining for IL-13 and IFN-γ in CD4⁺CD3⁺T cells in cell suspensions from breast cancer tumors. Staining is specific as it can be inhibited by adding recombinant human IL-13 (b, middle panel). FIG. 2c shows that CD3+CRTH2+ T cells (white) can be detected in breast cancer tumor sections. FIG. 2d shows the correlation between the frequency of IL-13-expressing CD4⁺T cells by flow cytometry and IL-13 secretion to tumor supernatants.

FIG. 3: IL-13 in breast cancer tumors.

FIGS. 4a to 4e show that human breast cancer tumors developed in humanized mice are infiltrated with DCs. 10×10⁶ tumor cells are inoculated subcutaneously into the flank of humanized mice four weeks post-CD34⁺HPC-transplant. FIG. 4a shows the comparative tumor size of at 4 days after inoculation. FIG. 4b shows representative kinetic of tumor development in humanized mice implanted with Hs578T breast cancer cells. FIG. 4c is a representative FACS analysis of tumor cell suspension: staining with HLA-DR (ordinate) and Lineage (abscissa) mAbs (left plot). Staining with CD123 (ordinate) and CD11c (abscissa) and analysis of reciprocal expression by pDC (CD123⁺CD11c⁻) and mDC (CD123⁻CD11c⁺) in gate for lineage negative HLA-DR⁺ cells (right plot). FIG. 4d shows the DC infiltration in lymph nodes draining breast cancer tumors and in contralateral lymph nodes. Percentage of HLA-DR⁺Lin⁻ cells (ordinate). N=9 mice/group analyzed within the same experiment. Wilcoxon test. FIG. 4e shows the HLA-DR (green) and DC-LAMP (red) staining on frozen tissue sections (10×/0.40 upper and 40×/1.25 lower panels). N=10 mice bearing Hs578T breast cancer were analyzed.

FIGS. 5a through 5e show that the reconstitution with CD4⁺T cells is associated with accelerated early development of breast cancer tumors. PBS (100 μl), autologous CD8⁺T cells (10×10⁶ cells/100 μl PBS) alone or together with CD4⁺T cells (10×10⁶ cells/100 μl PBS) are injected at day 3, 6 and 9 post-tumor implantation into (FIG. 5a) Hs578T breast cancer tumor (n=13, 6 and 15 studies with a total of n=36 [PBS], 21 [CD8⁺T cells], and 55 [CD4⁺ and CD8⁺T cells] humanized mice, respectively); kinetics of tumor development in representative cohort of mice bearing Hs578T tumors (left) or MCF-7 tumors (right). FIG. 5b shows the respective tumor size at day 12 in all mice, each dot represents one mouse. FIG. 5c shows the CD4⁺T cells were purified from the breast cancer tumor of donor OncoHumouse 15 days after T cell reconstitution. T cells from 4-6 mice were pooled and 1.5×10⁶ cells were injected once into tumors of recipient autologous OncoHumouse. Control mice received PBS. Tumor size at day 11. n=4 mice in two independent experiments. One-sided paired t-test. FIGS. 5d and 5e show the vascularization of the tumors.

FIGS. 6a and 6b show that accelerated breast cancer development requires CD4⁺T cells and autologous DCs. In FIG. 6a, PBS (100 μl), immature monocyte-derived DCs generated in cultures with GM-CSF and IL-4 (1×10⁶ cells/100 μl PBS), autologous CD4⁺T cells (10×10⁶ cells/100 μl PBS) or both were transferred into Hs578T breast cancer tumor in NOD-SCID β₂m^{-/-} mice without CD34⁺HPC transplant. FIG. 6b shows the kinetics of tumor development (n=3 mice/group).

FIGS. 7a and 7b show IL-13 expressing CD4⁺T cells in breast cancer tumors in humanized mice. In FIGS. 7a and 7b autologous CD4⁺T cells (10×10⁶ cells/100 μl; with or without CD8⁺T cells) were injected into Hs578T breast cancer tumors in OncoHumouse at days 3, 6, and 9 post-tumor implantation. At day 15, CD4⁺T cells were purified from tumor (FIG. 7a and FIG. 7b) and LN (FIG. 7a). FIG. 7a shows the cytokine secretion by Multiplex Bead Analysis after overnight restimulation with PMA lonomycin (4 studies). FIG. 7b shows the intracellular cytokine staining (representative of 3 studies, n=10 mice).

FIGS. 8a and 8b show that the breast cancer microenvironment modulates mDCs to induce CD4⁺T cells secreting type 2 cytokines. In FIG. 8a, HLA-DR⁺Lin⁻ DCs were sorted from LNs draining Hs578T breast tumors (day 4, 8 mice/group). Intracellular cytokine expression by CD4⁺T cells primed with DCs sorted from day 4 tumors and restimulated for 5 h with PMA ionomycin in presence of brefeldin A (FIG. 8b). Dot plots are gated on CD3⁺T cells. Representative of n=8 mice.

FIGS. 9a to 9c shows the accelerated breast cancer development can be inhibited with IL-13 antagonists. In FIG. 9a, PBS (100 μl) or autologous CD4⁺ & CD8⁺T cells (10×10⁶ cells/100 μl PBS) were injected into Hs578T breast cancer tumors in OncoHumouse at days 3 and 6 after tumor implantation. Isotype control or a mixture of anti-IL-13 antibody and rhIL-13Rα2/Fc chimera (100 μg/injection) were administrated at days 4, 6 and 8 post-tumor implantation (2 studies, 6 OncoHumouse/group with T cells, 5 Oncohumice in PBS control group); average and SEM. FIG. 9b shows the kinetics of tumor development. In FIG. 9b is the same as FIG. 9a, but single mice were analyzed at day 13. FIG. 9c is the same FIG. 9b, but mice were injected only with anti-IL-13 antibody. Paired t-test.

FIG. 10 is a graph that shows that prostate cancer tumors reconstituted with CD4+T cells but not control, showed high levels of IL-13 in supernatants.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, Type II cytokine antagonists are used to described active agents that interfere, reduce or eliminate the activity of cytokines that normally would aid in the activation of a Type 2 helper T cell, e.g., anti-IL-4, IL-5, IL-9, IL-13 or IL-25 antibody, a humanized anti-IL-4, IL-5, IL-9, IL-13 or IL-25 antibody; inactivated IL-4, IL-5, IL-9, IL-13 or IL-25 and combinations thereof; soluble IL-4, IL-5, IL-9, IL-13 or IL-25 receptors and combinations thereof, as well as, combinations of two or more of the above.

As used herein, the term “oncohumammal” is used to refer to non-human mammal that is immune deficient into which a human immune system has been grafted and to which a human cancer has been implanted. As will be apparent to the skilled artisan, a number of existing animals may be used as the immune deficient animal. Also, a number of methods for the non-lethal manufacturing of immune deficient animals is available, including non-lethal doses of radiation, chemical treatments, animals with one or more genetic mutations, the genetic manipulation of the mammal by the making of a transgenic, a knock-out, a conditional knock-out, a knock-in and the like. One example of an “oncohumammal” is an “oncohumouse,” in which a mouse is used as the platform for the introduction of at least a portion of a human immune system and a human tumor. The tumor may one or more primary tumors (e.g., autologous with the immune system implanted, i.e., from the same patient), one or more tumor cell clones and/or one or more tumor cell lines.

Immune Deficient Animal Hosts. Any immunodeficient mammal may be used to generate the animal models described herein. As used herein, the term “immunodeficient” is used to describe an alteration that impairs the animal's ability to mount an effective immune response. As used herein, an “effective immune response” is used to describe a human immune response in the host animal that is capable or, e.g., destroying invading pathogens such as (but not limited to) viruses, bacteria, parasites, malignant cells, and/or a xenogeneic or allogeneic transplant. One example of an immunodeficient mammal is the immunodeficient mouse referred to as a severe combined immunodeficient (SCID) mouse, which generally lacks recombinase activity that is necessary for the generation of immunoglobulin and functional T cell antigen receptors, and thus does not produce functional B and T lymphocytes.

Immune deficient mice, rats or other animals may be used, including those that are deficient as a result of a genetic defect, which may be naturally occurring or induced. For example, heterologous or homologous: nude mice, immunodeficient nonobese diabetic/LtSz-scid/scid (NOD/SCID) mice with additional mutation in β2-microglobulin gene (NOD/SCID/β2m^{-/-}), Rag 1^{-/-}, Rag 2^{-/-} mice and/or PEP^{-/-} mice, mice that have been cross-bred with these mice and have an immunocompromised background may be used for implanting or engrafting a human immune system and/or cells as described herein. The deficiency may be, for example, as a result of a genetic defect in recombination, a genetically defective thymus or a defective T-cell receptor region, NK cell defects, Toll receptor defects, Fc receptor defects, immunoglobulin rearrangement defects, defects in metabolism, combinations thereof and the like. Induced immune deficiency may be as a result of administration of an immunosuppressant, e.g. cyclosporin, NK-506, removal of the thymus, radiation and the like.

Various transgenic immune deficient mice are currently available or can be mated or cross-bred and selected in accordance with conventional techniques. Generally, the immune deficient mouse will have a defect that inhibits maturation of lymphocytes, particularly lacking the ability to rearrange immunoglobulin and/or T-cell receptor regions, Toll receptors, and the like. Female, male, castrated or uncastrated mice may be used depending on the effect of the availability of, e.g., androgens, on the course of the tumor growth. In addition to mice, immune deficient rats or similar rodents may also be employed in the practice of the invention.

As used herein, the term “compounds,” “agent(s),” “active ingredient(s),” “pharmaceutical ingredient(s),” “active agents,” “bioactive agent” are used interchangeably and defined as drugs and/or pharmaceutically active ingredients. The present invention may use or release of, for example, any of the following drugs as the pharmaceutically active agent in a pool of test compounds to isolate one or more lead compounds. A number of test compounds may be tested, isolated and purified using the methods of the present invention.

Examples of test compounds include, antitumor agents, anti-miotics, steroids, sympathomimetics, local anesthetics, antimicrobial agents, antihypertensive agents, antihypertensive diuretics, cardiotonics, coronary vasodilators, vasoconstrictors, β-blockers, antiarrhythmic agents, calcium antagonists, anti-convulsants, agents for dizziness, tranquilizers, antipsychotics, muscle relaxants, respiratory agents, non-steroidal hormones, antihormones, vitamins, herb medicines, antimuscarinic, muscarinic cholinergic blocking agents, mydriatics, psychic energizers, humoral agents, antispasmodics, antidepressant drugs, anti-diabetics, anorectic drugs, anti-allergenics, decongestants, antipyretics, antimigrane, anti-malarials, anti-ulcerative, peptides, anti-estrogen, anti-hormone agents, antiulcer agents, anesthetic agent, drugs having an action on the central nervous system or combinations thereof. Additionally, one or more of the following bioactive agents may be combined with one or more carriers and the present invention (which may itself be the carrier).

Different lineages of immune-compromised mice may used in conjunction with the present invention. In one embodiment, the immune-compromised mouse may be made transgenic with one or more genes that are tumor suppressors, cytokines, enzymes, receptors, or even inducers of apoptosis. Alternatively, the second gene may be derived from an oncogene. Examples of oncogene include ras, myc, neu, raf erb, src, fms, jun, trk, ret, gsp, hst, bcl and abl. Genes may also include a tumor suppressor, the tumor suppressor may be, e.g., p53, p16, p21, MMAC1, p73, zac1, BRCAI and Rb. Other genes may tumor cytokine, the cytokine is selected from the group consisting of IL-2, IL-2, IL-3, IL-4, IL-5, IL-6, IL-7, IL-8, IL-9, IL-10, IL-11, IL-12, IL-13, IL-14, IL-15, TNF, GMCSF, β-interferon and γ-interferon. In other embodiments the gene may be an enzyme, e.g., cytosine deaminase, adenosine deaminase, .beta.-glucuronidase, hypoxanthine guanine phosphoribosyl transferase, galactose-1-phosphate uridyltransferase, glucocerbrosidase, glucose-6-phosphatase, thymidine kinase and lysosomal glucosidase. In other embodiments, the gene may be a receptor, e.g., CFTR, EGFR, VEGFR, IL-2 receptor and the estrogen receptor. In other embodiment, the gene may be an inducer of apoptosis, e.g., Bax, Bak, Bcl-X.sub.s, Bik, Bid, Bad, Harakiri, Ad E1B and an ICE-CED3 protease. In certain embodiments, the cells that are made transgenic and/or transfected are human cells that are implanted in the mouse.

The present invention further provides a method of enhancing the effectiveness of ionizing radiotherapy by administering, to a tumor site in a mammal, an anti-angiogenic factor protein prior to radiation therapy; and ionizing radiation, wherein the combination of anti-angiogenic factor administration and radiation is more effective than ionizing radiation alone.

The present invention also includes pools and/or leads of therapeutic compounds in, e.g., a pharmaceutically acceptable carrier or diluent. With respect to in vivo applications, the compounds identified by screening methods may be administered to the oncohumouse in a variety of ways including, for example, parenterally, orally or intraperitoneally. Parenteral administration includes administration by the following routes: intravenous, intramuscular, interstitial, intraperitoneal, intradural, epidural, intraarterial, subcutaneous, intraocular, intrasynovial, transepithelial, including transdermal, pulmonary via inhalation, opthalmic, sublingual and buccal, topical, including ophthalmic, dermal, ocular, rectal, vaginal and nasal inhalation via insufflation or nebulization.

The Type II cytokine antagonists (and others) may be orally administered, for example, with an inert diluent or with an assimilable edible carrier, they can be enclosed in hard or soft shell gelatin capsules, or they can be compressed into tablets. For oral therapeutic administration, the active compounds can be incorporated with an excipient and used in the form of ingestible tablets, buccal tablets, troches, capsules, sachets, lozenges, elixirs, suspensions, syrups, wafers, and the like. The pharmaceutical composition may include active compounds in the form of a powder or granule, a solution or suspension in an aqueous liquid or non-aqueous liquid, or in an oil-in-water or water-in-oil emulsion.

The tablets, troches, pills, capsules and the like can also contain, for example, a binder, such as gum tragacanth, acacia, corn starch or gelatin. Excipients, such as dicalcium phosphate, a disintegrating agent, such as corn starch, potato starch, alginic acid and the like, a lubricant, such as magnesium stearate, and a sweetening agent, such as sucrose, lactose or saccharin, or a flavoring agent may also be included. When the dosage unit form is a capsule, it may include a liquid carrier. Various other materials may be present as coatings or to otherwise modify the physical form of the dosage unit. For instance, tablets, pills, or capsules can be coated with shellac, sugar or both. A syrup or elixir may include the active compound, sucrose as a sweetening agent, methyl and propylparabens as preservatives, a dye and flavoring. Any material used in preparing any dosage unit will generally be pharmaceutically pure and substantially non-toxic. The active compound may be incorporated into sustained-release preparations and formulations.

The Type II cytokine antagonists (and others) may be administered parenterally or intraperitoneally. Solutions of the compound as a free base or a pharmaceutically acceptable salt may be prepared in water mixed with a suitable surfactant, e.g., hydroxypropylcellulose. Dispersions can also be prepared in glycerol, liquid polyethylene glycols, and mixtures thereof, and in oils. Under ordinary conditions of storage and use, these preparations can contain a preservative and/or antioxidants to prevent the growth of microbes and/or chemical degeneration.

The pharmaceutical forms of the Type II cytokine antagonists (and others) may be prepared for injectable use by including sterile aqueous solutions or dispersions and sterile powders for the extemporaneous preparation of sterile injectable solutions or dispersions. For intravenous or other like use, the compounds are generally sterile and may be provided in liquid suspension and/or resuspended for delivery via syringe. It can be stable under the conditions of manufacture and storage and must be preserved against the contaminating action of microorganisms, such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained by the use of a coating, e.g., lecithin, and incorporation into a particle of the required size (in the case of a dispersion) and by the use of surfactants as is well known to the skilled artisan. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride may be used.

Sterile injectable solutions are prepared by incorporating the Type II cytokine antagonists (and others) in the required amount in the appropriate solvent with various other ingredients enumerated above, as required, followed by filtered sterilization. Generally, dispersions are prepared by incorporating various sterilized active ingredients into a sterile vehicle that includes the basic dispersion medium and any of the other, ingredients from those enumerated above. In the case of sterile powders for the preparation of sterile injectable solutions, methods of preparation may include, e.g., vacuum drying, freeze-spraying, heat-vacuum and/or freeze drying techniques. Pharmaceutical compositions that are suitable for administration to the nose or buccal cavity include, e.g., powder, self-propelling and spray formulations, such as aerosols, atomizers and nebulizers.

The therapeutic Type II cytokine antagonists (and others) of this invention may be administered to a mammal alone or in combination with pharmaceutically acceptable carriers or as pharmaceutically acceptable salts, the proportion of which is determined by the solubility and chemical nature of the compound, chosen route of administration and standard pharmaceutical practice. The compositions may also include other therapeutically active compounds that are usually applied in the treatment of the diseases and disorders, e.g., cancer. Treatments using the present compounds and other therapeutically active compounds may be simultaneous or by intervals.

The in vivo analysis disclosed herein is based on immunodeficient mice reconstituted with CD34⁺HPCs and human tumors might prove useful in the analysis of the human immune system and human cancer. Immunodeficient mice implanted with human tumor xenografts, with or without adoptively transferred human peripheral blood leukocytes (PBL) (Mosier et al., 1988), have been used for many years as models to study human cancer (Mueller and Reisfeld, 1991; Reddy et al., 1987). They have permitted the identification of clinically relevant prognostic factors in hematological malignancies (Kamel-Reid et al., 1991; Kamel-Reid et al., 1989; Palucka et al., 1996; Uckun et al., 1995); and have played a significant role in pre-clinical development of cancer drugs and immune therapies (Bankert et al., 2001; Kelland, 2004). Such models have also helped in assessing the mechanisms of metastasis in solid tumors (Muller et al., 2001).

A major limitation of earlier models was the lack of human immune microenvironment. Indeed, approaches that were undertaken include: (i) tumor xenografts in the absence of human immune cells (Bankert et al., 2001; Mueller and Reisfeld, 1991; Reddy et al., 1987); (ii) xenografts of tumor surgical biopsies, which harbor immune cells that have been imprinted by the tumor (Anderson et al., 2003; Iwanuma et al., 1997; Sakakibara et al., 1996); and (iii) xenografts with human peripheral blood lymphocytes (SCID-huPBL) (Mosier et al., 1988), which are nearly devoid of human DCs. The approach used herein, mice engrafted with human CD34⁺HPCs and reconstituted with human immune system from a healthy volunteers, permits the analysis for the first time of early events in the biology of cancer cells implanted in the environment of human immune cells, which have not been previously exposed to tumor.

Generation of humanized mice: CD34⁺HPCs were obtained from apheresis of adult healthy volunteers mobilized with G-CSF and purified as previously described (Palucka et al., 2003). CD34⁻ fraction of apheresis was Ficoll-purified, obtained PBMCs were stored frozen and used as a source of autologous T cells. 2.5×10⁶ CD34⁺HPCs were transplanted intravenously into sublethally irradiated (12 cGy/g body weight of ¹³⁷Cs γ-irradiation) NOD-SCID β₂m^{-/-} mice (Jackson Laboratories). 10×10⁶ breast cancer cells (Hs578T, MCF7, 1806) harvested from long term cultures, were injected subcutaneously into the flank of the mice or in the mammary glands area. For experiments with NOD-SCID β₂m^{-/-} mice, they were sublethally irradiated (12 cGy/g body weight of ¹³⁷Cs γ-irradiation) the day prior to tumor implantation. Tumor size was monitored every 2-3 days. Tumor volume (ellipsoid) was calculated using the formula: (short diameter)²× long diameter/2.

Monocyte-derived dendritic cells and T cell purification: Monocyte-derived dendritic cells were generated from adherent fraction of PBMCs by culturing with GM-CSF (100 ng/ml) (Immunex, Seattle, Wash.) and IL-4 (25 ng/ml) (R&D systems, Minneapolis, Minn.). CD4⁺ and CD8⁺T cells were positively selected from thawed PBMCs using magnetic selection according to manufacturer instructions (Myltenyi Biotec, Auburn, Calif.). The purity was routinely >90%.

Immunofluorescence: Tissues were frozen in Tissue-Tek (OCT, Allegiance, McGaw, Ill.), cryosectioned on Superfrost Plus slides (Fisher scientific, Pittsburgh, Pa.) and fixed with cold acetone. Direct staining: HLA-DR FITC (BD Pharmingen, San Diego, Calif.); CRTH2-PE; IL-13-PE; CD3-FITC. Indirect staining: DC-LAMP (Immunotech, Marseille, France) following by anti-mouse IgG conjugated to Texas-Red (Jackson Immunoresearch, West Grove, Pa.). Confocal microscopy was performed using a Leica TCS-NT SP (Leica, Deerfield, Ill.). To assess tumor vascularization, OncoHumouse were injected with FITC-lectin (150 microliters at 2 mg/ml) (Vector Laboratories, Burlingame, Calif.) intravenous (iv) 10 min later mice were anesthetised and infused with PFA 4% iv. 10 μm tumor sections were fixed and mounted in Vectashield with DAPI (Vector Laboratories, Burlingame, Calif.) and analyzed with Olympus BX51 equipped with planapo objectives and Photometrics coolsnaps HQ and Metamorph software (UIC).

Flow cytometry: Cell suspensions were obtained from tumor, lymph nodes, and spleens by digestion with collagenase D (2 mg/ml) (Roche Diagnostics, Indianapolis, Ind.) 45 min at 37° C. Bone marrow cells were washed out of the harvested bones. Cell suspensions were washed twice and stained in PBS 2 mM EDTA 5% AB serum using the following antibodies: Lin, CD45, IgD, CD80-FITC, CD123, CD86, HLA-ABC, CD3-PE, HLA-DR-PerCP, CD11c, CD14, CD19-APC (BD Pharmingen, San Diego, Calif.) and CD40-PE (Immunotech, Marseille, France).

T cell cytokines: Naive CD4⁺T cells were obtained from buffy coats after magnetic depletion using CD8, CD14, CD19, CD16, CD56 and glycophorine A microbeads (Miltenyi Biotec, Auburn, Calif.) and sorted based on the CD4⁺CCR7⁺CD45RA⁺ phenotype. NKT cells were depleted by exclusion of Vα24⁺ CD4⁺T cells from the sort gate. DCs were sorted based on HLA-DR⁺Lin-CD 11c⁺ and HLA-DR⁺Lin-CD123⁺ phenotype. Naive CD4⁺T cells (5×10⁴/well) were cultured with DC (5×10³/well) in RPMI 1640 supplemented with 10% human AB serum (Gemini BioProducts, Woodland, Calif.). To assess cytokine secretion by Luminex, T cells were harvested at day 5, washed twice, resuspended at a concentration of 1×10⁶/ml and restimulated for 16 h with PMA (50 ng/ml) and ionomycin (1 μg/ml) (Sigma, St. Louis, Mo.). To assess cytokine expression by intracellular staining, T cells were harvested on day 6 of the culture, washed twice and restimulated 5 hours with PMA and ionomycin. Brefeldin A (10 mg/ml) (BD Pharmingen, San Diego, Calif.) was added for the last 2.5 hours. T cells were labeled with anti-CD3 and Abs to IL-4, IL-13, TNF, IFN-γ and IL-2 (BD Pharmingen, San Diego, Calif.).

In vivo IL-13 blocking: mice were injected intratumorally at day 4, 6 and 8 post-tumor implantation with anti-IL-13 mAbs and rhIL-13Rα2/Fc chimera or goat IgG isotype control (100 μg/ml each) (R&D systems, Minneapolis, Minn.).

Tumor samples from patients diagnosed with infiltrating or invasive breast carcinoma were obtained Baylor University Medical Center Tissue Bank (IRB#005-145). Samples were minced into small fragments and digested in a triple enzyme mix containing collagenase 2.5 mg/ml, hyaluronidase 1 mg/ml, DNase 20 U/ml 2-3 hours at 37° C. The suspension was filtered, washed, obtained cells were resuspended at a concentration of 1×10⁶/ml and activated with PMA (50 ng/ml) and ionomycin (1 μg/ml) (Sigma, St Louis, Mo.) for 16 h. Cytokine production was analyzed in the culture supernatant by Luminex. Alternatively, whole tumor fragments were placed in the overnight culture with PMA and lonomycin. For intracellular cytokine staining, cells were stimulated 5 hours with PMA and ionomycin. Brefeldin A (10 mg/ml) (BD Pharmingen, San Diego, Calif.) was added for the last 2.5 hours. Cells were labeled with anti-CD3 and anti-CD4 mAb and intracellular cytokine staining was performed using Abs to IL13 and IFNy (BD Pharmingen, San Diego, Calif.). For inhibition of IL13 staining, anti-IL13 mAb was incubated with recombinant human IL13 (5 μg/ml) for 1 hour at room temperature prior use. Cells were fixed in PFA 1% and analyzed by flow cytometry. A piece of each tissue was frozen in for immunofluorescence analysis. Sections were labeled with CD3 Alexa 488 mab (BD Pharmingen, San Diego, Calif.) and mounted with DAPI.

Statistics: Parametric t-test and non-parametric Mann-Whitney and Wilcoxon tests were used to assess the significance of observed differences. Parametric Pearson Correlation and non-parametric Spearman correlation were used as indicated.

It was found that breast cancer tumors are infiltrated with mature dendritic cells (DCs), which often are engaged in tight clusters with CD4⁺T cells. It was found that, in the breast cancer tumor microenvironment, CD4⁺T cells secreting type 1 (IFN-γ) and type 2 (mostly IL-13) cytokines were found. Immunofluorescence staining on frozen tissue sections revealed IL-13 expression on breast cancer cells. To demonstrate the link between breast cancer, DCs and CD4⁺T cell polarization NOD/SCID β2m^{-/-} mice engrafted with human CD34⁺ hematopoietic progenitor cells (HPCs) and T cells from healthy volunteers and implanted with human breast cancer cell lines were used. There, breast cancer cells attract human DCs and imprint them to prime naïve CD4⁺T cells to secrete IL-13. CD4⁺T cells promote tumor development, which can be inhibited with IL-13 antagonists. Thus, breast cancer promotes skewed DC maturation to elicit pro-cancer immunity.

It has been found that breast cancer is rich in certain subsets of human DCs (Bell et al., 1999). These include large quantities of immature myeloid DC (mDC) subsets such as Langerhans cells and interstitial DCs. The presence of these cells per se might not be surprising. Indeed, it is the function of immature DCs to monitor epithelial surfaces. Therefore, a tumor might use the mechanisms of physiological tissue homeostasis such as infiltration with immature DCs. Interestingly, peri-tumoral areas of breast cancer tissue display mature DC-LAMP+ DCs, which under normal physiological conditions can only be found in lymphoid tissues. Presence of mature DC outside lymphoid organs is linked with inflammation and can be observed in aseptic synovial inflammation in rheumatoid arthritis (Radstake et al., 2005; Thomas et al., 1999) Radstake et al., 2005; (Gordon and Taylor, 2005) or in the blood of patients with systemic autoimmunity (Banchereau et al., 2004; Blanco et al., 2001). However, the immunological consequences of the presence of mature DC in tumors remain unknown.

The characteristics of CD4⁺T cells infiltrating breast cancer tissue samples from patients were determined. It was found that IL-13 is found in the breast cancer microenvironment and IL-13 staining on breast cancer cells. These observations prompted us to engage into mechanistic studies. However, studies in humans are hampered by limited availability of samples making it difficult to establish causative links. Therefore, a model of humanized mice was made in which (Palucka et al., 2003) breast cancer cell lines were also grafted into the a humouse. These mice may be immunodeficient nonobese diabetic/LtSz-scid/scid (NOD/SCID) β2 microglobulin-deficient (NOD/SCID/β2m^{-/-}) mice transplanted with human CD34⁺ hematopoietic progenitor cells (CD34⁺HPCs) (Humouse). These mice develop the components of human innate immune system such as macrophages, all subsets of human DCs and component of human adaptive immune system, B cells (Palucka et al., 2003). Grafting autologous T cells permits us to analyze modulation of human T cell subsets. It is shown herein that DCs that infiltrate breast cancer tumors polarize naive CD4+T cells towards secretion of IL-13.

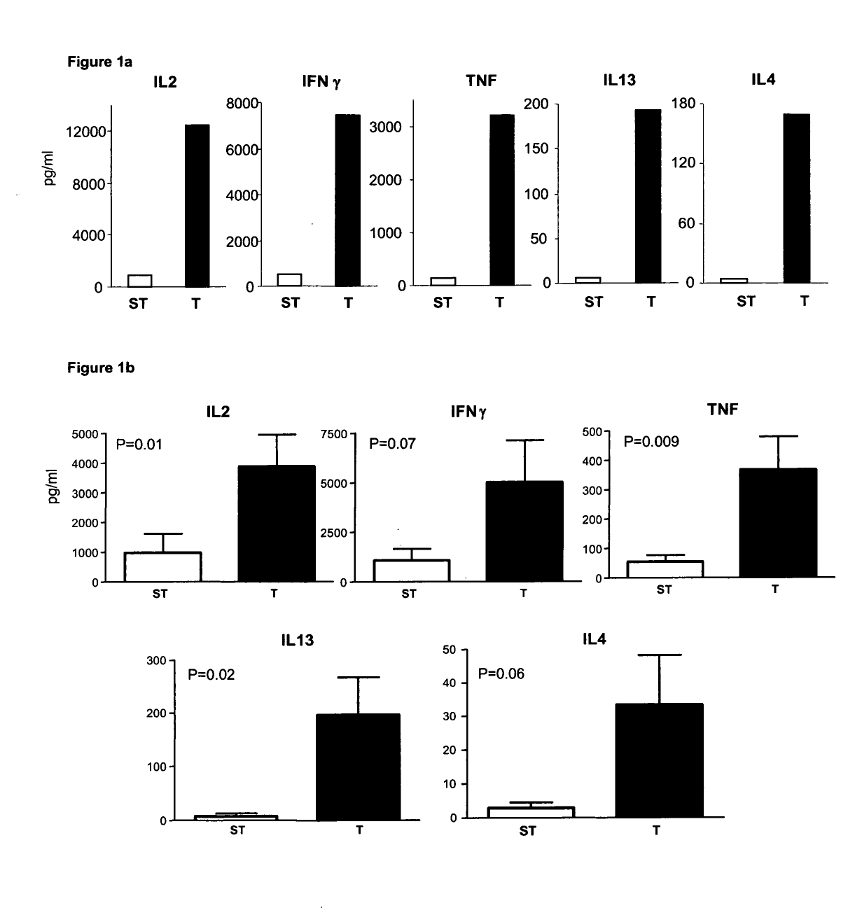

Microenvironment of breast cancer tumors from patients is rich in type 2 cytokines. Breast cancer tumors are infiltrated, in peri-tumoral areas, with mature DCs that are engaged in tight clusters with T cells (Bell et al., 1999) suggesting an ongoing immune response. To investigate the consequences of this interaction for breast cancer tumor microenvironment, the pattern of T cell cytokines in tumor biopsies from patients with breast cancer was analyzed. Samples from 21 patients were analyzed, which included in situ and invasive duct and/or mucinous carcinoma of the breast as well as lobular carcinoma. Whenever possible, tumor sites as well as surrounding tissue (macroscopically uninvolved) obtained from the same patient were analyzed. Single cell suspensions (FIG. 1a) or whole tumor fragments (FIG. 1b) were activated for 16 hrs with PMA/Ionomycin and supernatants were assayed by Cytokine Bead Array.

Analysis of single cell suspensions in the initial cohort of patients (Pt#1-6 in Table 1) demonstrated high levels of IL-2, IFN-γ and TNF in tumor samples, but not surrounding tissues (FIG. 1a and Table 1). Furthermore, high levels of IL-13 (200 pg/ml) and IL-4 (180 pg/ml), could be detected in three out four evaluable samples suggesting Th2 polarization. To exclude the possibility that tissue processing might skew the data, in the next cohort of patients, cytokines whole tumor fragments were activated to analyze the cytokine pattern. As shown in FIG. 1b and Table 1, high levels of IL-2 and IFN-γ were found in all samples (13/14); IL-13 in 11/13 samples and IL-4 in 7/13 samples (FIG. 1b and Table 1). The levels of IL-2, TNF and IL-13 were significantly higher in supernatants from tumor sites than in supernatants from tumor surrounding tissue (FIG. 1b). Thus, the microenvironment of breast cancer samples from patients is rich in IFN-γ and in type 2 cytokines, suggesting T cell polarization.

TABLE 1
Type 1 and Type 2 cytokines in the microenvironment
of breast cancer tumors from patients.

| Patient | | | | | |
|---|---|---|---|---|---|
| cohort | IL-2 pg/ml | IFN-γ pg/ml | TNF pg/ml | IL-13 pg/ml | IL-4 pg/ml |
| #1-6 Tumor | Mean ± SEM | 8365 ± 2721 | 5619 ± 2118 | 356 ± 286 | 176 ± 95 |
| cell suspension | Range N | 1033-12500 | 1679-11265 | 10-1204 | 6-443 |
| 4/6 samples | 9574 ± 2926 | 4/4 | 4/4 | 2/4 | 3/4 |
| evaluable | 796-12500 | | | | |
| | 4/4 | | | | |
| #7-21 Whole | 3867 ± 1067 | 5005 ± 2124 | 367 ± 112 | 196 ± 71 | 33 ± 15 |
| tumor fragment | 75-13022 | 124-27599 | 0-1192 | 0-930 | 0-199 |
| 13/14 samples | 13/13 | 13/13 | 11/13 | 11/13 | 7/13 |
| evaluable | | | | | |

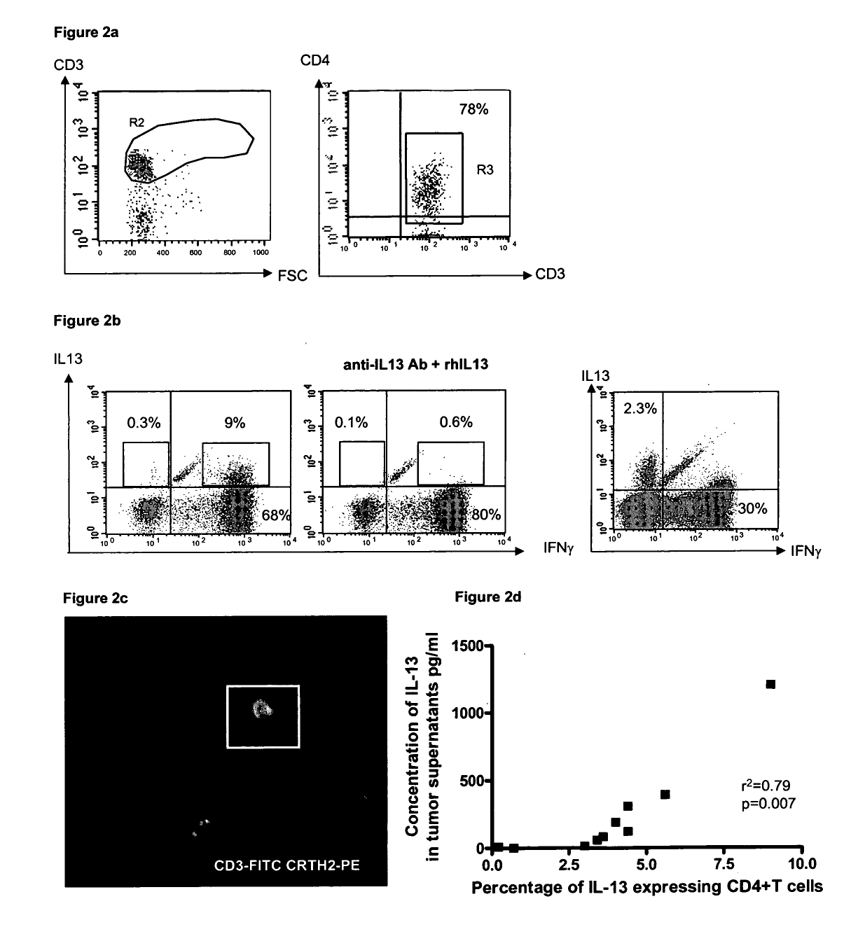

Breast cancer tumors from patients are infiltrated with CD4⁺T cells secreting type 1 and type 2 cytokines. To determine whether T cells actually contribute to the production of cytokines detected in breast cancer microenvironment, the T cell composition and the cytokine expression pattern in single cell suspensions was determined. Flow cytometry indicate the prevalence of CD4⁺T cells (≧75%; FIG. 2a). Limited amount of tissue available for analysis prompted us to initially focus on two cytokines, i.e., IFN-γ (type 1 cytokine) and IL-13 (type 2 cytokine whose levels in supernatants analysis were higher than that of IL-4). Intracellular staining demonstrated the presence of IL-13 expressing CD4⁺T cells, which could represent up to 9% of CD4⁺T cells (FIG. 2b). The staining was specific as it could be blocked by excess recombinant IL-13 (FIG. 2b). Interestingly, two types of staining in different tumor samples were observed, i.e., double positive T cells expressing both IL-13 and IFN-γ, and single positive T cells expressing either IL-13 or IFN-γ (FIG. 2b), the latter one consistent with the classical definition of T cell polarization (Mosmann and Coffman, 1989). In line with is, T cells expressing chemoattractant receptor-homologous molecule expressed on Th2 cells (CRTH2) (Nagata et al., 1999); (Cosmi et al., 2000) could be detected by immunofluorescence on frozen tissue sections from some tumors (FIG. 2c). The mean frequency of IL-13-expressing CD4⁺T cells in 11 tumor samples analyzed was 3.7%±SEM 0.7%, range 0.2%-9.3%. The frequency of CD4⁺CD3⁺T cells demonstrating intracellular expression of IL-13 was highly correlated with IL-13 concentration in supernatants of tumor cells (r²=0.79, p=0.007, Pearson correlation, FIG. 2d). Thus, the microenvironment of breast cancer samples from patients is rich in CD4⁺T cells secreting type 1 and type 2 cytokines.

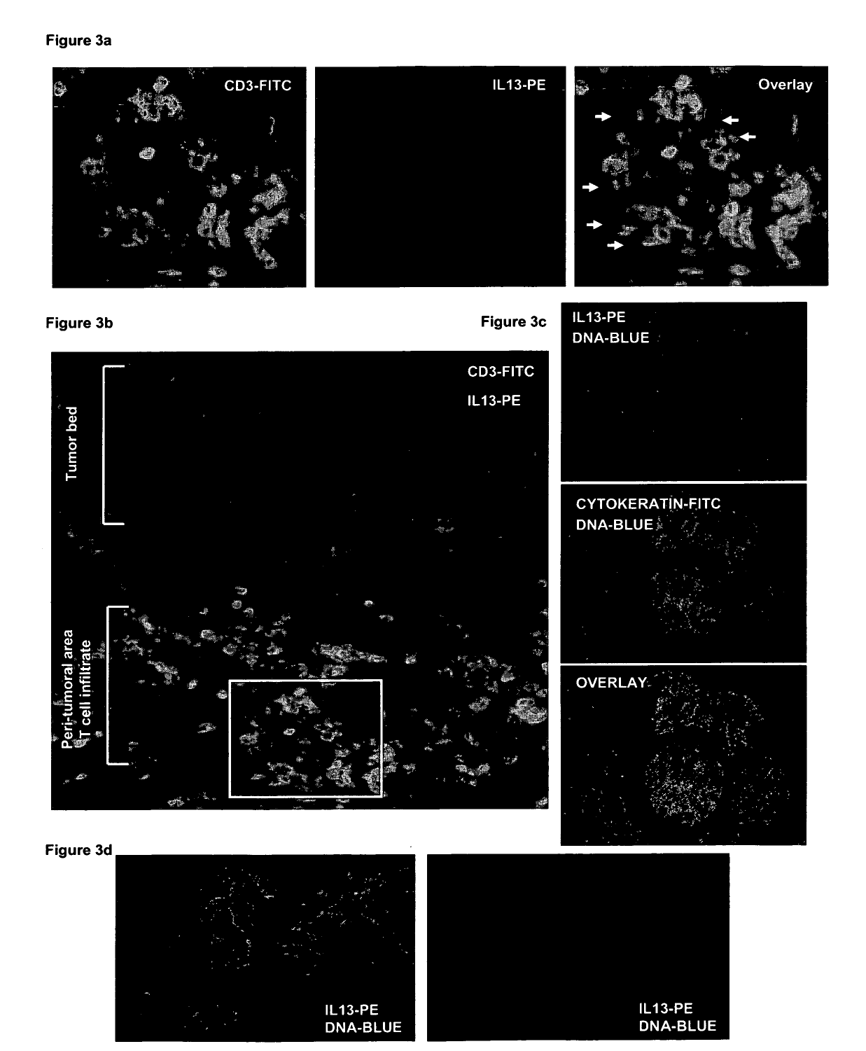

High level of IL-13 expression by breast cancer cells. Next, whether IL-13-expressing CD4⁺T cells are engaged in clusters with mature DCs infiltrating breast cancer tumors was analyzed. As shown in FIG. 3a, immunofluorescence on frozen breast cancer tissue sections demonstrated that some tumors infiltrates of CD3⁺T cells co-staining with anti-IL-13 mAb (FIG. 3a, yellow double positive cells in the overlay graph are indicated with white arrows). These CD3⁺T cells were located in peri-tumoral areas (FIG. 3a). However, in many cases this analysis was overwhelmed by high level of a homogenous IL-13 staining observed in tumor beds of 11 analyzed breast cancer tumor samples (FIG. 3b, red stained cells separate from the green T cell infiltrate). These cells co-stained with cytokeratin suggesting IL-13 expression by breast cancer cells (FIG. 3c). Finally, IL-13 staining was abolished by the excess of rhIL-13 (FIG. 3d) thus demonstrating specificity. Thus, the major population of cells, which expresses IL-13 in the microenvironment of breast cancer, are actually breast cancer cells.

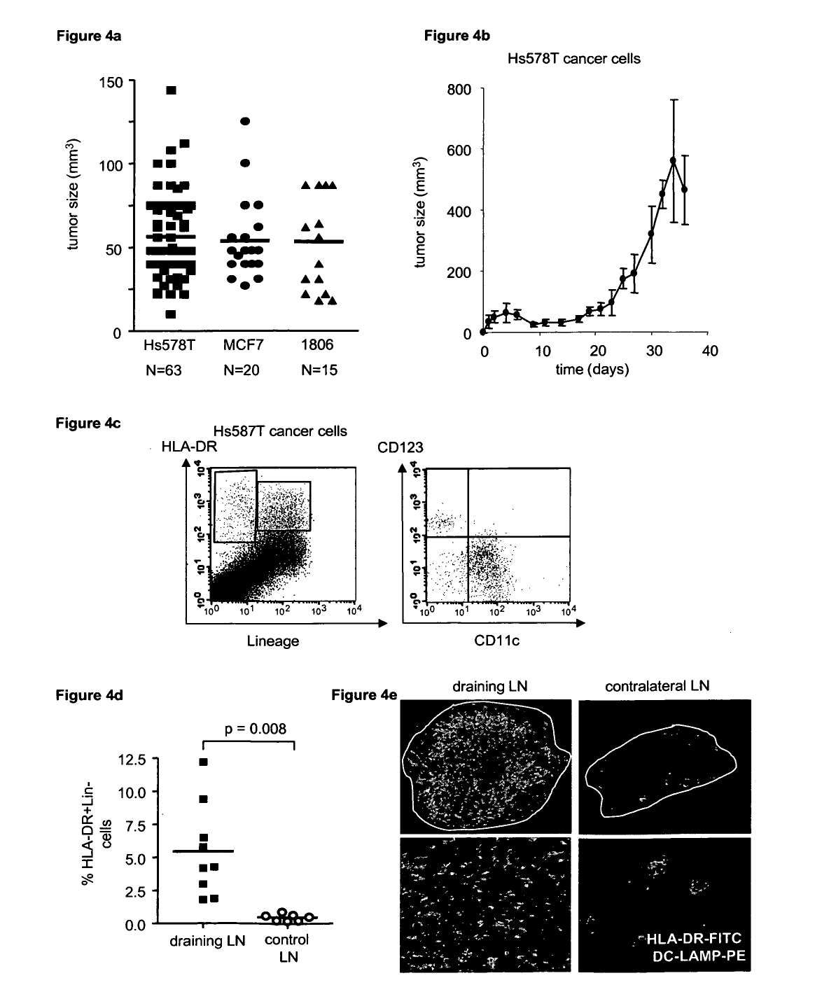

Breast cancer tumors in Humouse are infiltrated with human myeloid DCs. To demonstrate the link between breast cancer, DCs and CD4⁺T cell polarization an in vivo model of the human immune system and human breast cancer was developed and used. Sub-lethally irradiated adult NOD/SCID/β2m⁻ mice were transplanted with human G-CSF mobilized CD34⁺HPCs isolated from the healthy volunteer blood apheresis. At 4 weeks later when these mice develop the components of the human immune system including DCs and B cells (Palucka et al., 2003), 10⁷ human tumor cells were implanted subcutaneously (s.c.) into the flank. Three different breast cancer cell lines (Hs587T, MCF-7 and 1806) representing primary (Hs587T and 1806) and metastatic (MCF-7) tumors with different histopathological and phenotypic characteristics were tested (Table 2). At day 4, a clearly delineated tumor was measurable with three tested cell lines (FIG. 4a). Tumor development was bi-phasic (FIG. 4b) with a ten-day tumor establishment phase followed by a temporary decrease in the tumor volume (Aspord et al. submitted). From day 20 on, Hs578T and 1806 tumors, which are estrogen-independent, progressed at the primary site (FIG. 4b) and animals developed distant metastasis (Aspord et al. submitted).

TABLE 2
Characteristics of breast cancer cell lines analyzed

| | Histology | |
|---|---|---|
| cell line | Origin | Phenotype |
| Hs578T | Primary | ER^{neg} |
| | Ductal | PR^{neg} |
| | carcinoma | Her2/neu^{low} |
| | | EGFR^{low} |
| MCF7 | Pleural | ER^{pos} |
| | effusion | PR^{pos} |
| | | Her2/neu^{low} |
| | | EGFR^{pos} |
| 1806 | Primary | ER^{neg} |
| | squamous | PR^{neg} |
| | carcinoma | Her2/neu^{neg} |

Single cell suspensions were prepared from tumors harvested at days 4 and 30 post-implant. Flow cytometry analysis was performed as illustrated in FIG. 4c. Hs578T tumors were infiltrated with HLA-DR⁺ cells that did not express lineage (Lin) markers of T cells, B cells, monocytes and NK cells (HLA-DR⁺ Lin⁻ cells) and thus comprise DCs (Pulendran et al., 2000) (% HLA-DR⁺ Lin⁻ cells in single cell suspension; mean±SD=0.62±0.13). The HLA-DR⁺ Lin⁻ cells contained HLA-DR⁺CD11c⁺ myeloid DCs and HLA-DR⁺CD123⁺ plasmacytoid DCs (FIG. 4c). The three tested breast cancer tumors (Hs578T, MCF7 and 1806) showed infiltration with DCs, 1806 tumors showed the lowest infiltrate among the three cell lines (Table 3). Infiltration with DCs increased with time in Hs578T tumors (Table 3). Finally, infiltration with DCs was specific to breast cancer and could not be seen in control mice who received s.c. PBS injection instead of tumor cell implant or in skin biopsies taken from the area just outside the implanted breast cancer tumor (Aspord et al. submitted). A similar pattern of DC attraction was found in orthotopically implanted tumors (not shown).

TABLE 3
DCs in breast cancer tumors and their draining
lymph nodes in the humanized mice model.

| | time post | | HLA-DR + Lin-cells (%) | | | HLA-ABC |
|---|---|---|---|---|---|---|
| | tumor | number | | draining | | (%) |
| cell line | implant | of mice | tumor | LN | control LN | BM |
| Hs578T | 4 days | 16 | 0.74 ± 0.06 | 15.34 ± 3.78 | 4.75 ± 1.71 | 65.67 ± 4.99 |
| MCF7 | 4 days | 4 | 0.78 ± 0.15 | 23.7 ± 2.6 | 4.45 ± 0.45 | 82.75 ± 2.56 |
| 1806 | 4 days | 3 | 0.22 ± 0.06 | 3 ± 1.9 | 0.88 ± 0.61 | 55.3 ± 9.35 |
| Hs578T | 30 days | 12 | 1.44 ± 0.2 | 2.79 ± 0.57 | 0.09 ± 0.01 | 58.71 ± 4.82 |
| 1806 | 30 days | 8 | 0.26 ± 0.03 | 12.45 ± 4 | 4.1 ± 1.01 | 56.00 ± 9.43 |

Lymph nodes draining breast cancer tumors also showed DC infiltration as illustrated in FIG. 4d with Hs578T tumors (% HLA-DR⁺ Lin⁻ cells in single cell suspension; mean±SD=5.46±1.16) (FIG. 4d and Table 2). The lymph nodes draining breast cancer tumors were infiltrated with human DCs co-expressing HLA-DR and DC-LAMP (FIG. 4e), a phenotype of mature DCs. In contrast, contralateral lymph nodes showed only few DC-LAMP expressing DC (FIG. 4e). Thus, similarly to the present inventors' findings in patients, breast cancer tumors grafted into humanized mice are rich in DCs and trigger their maturation.

CD4⁺T cells promote development of breast cancer tumors. To determine whether CD4⁺T cells will be polarized similarly to the findings in patients, humanized mice bearing human breast cancer tumors were reconstituted with T cells by intratumoral injection. The T cells were isolated from the blood mononuclear cells of the CD34⁺HPCs donor and were thus autologous to the Antigen Presenting Cells (APCs) that had developed after CD34⁺HPC transplant in vivo but allogeneic to the implanted tumor cells.

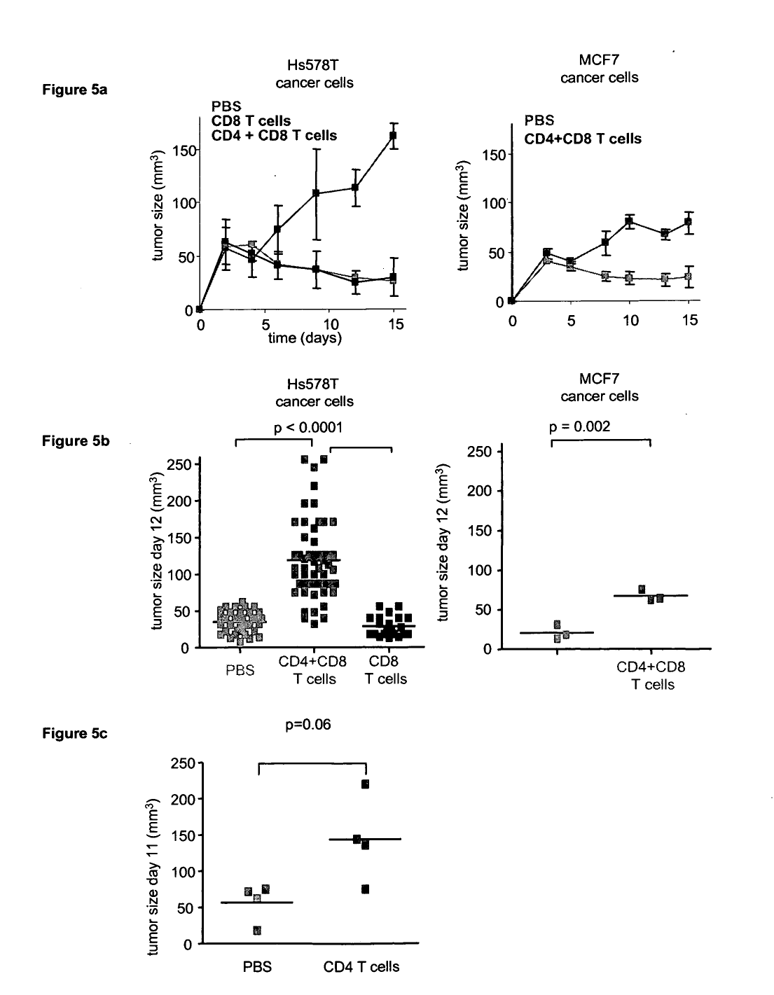

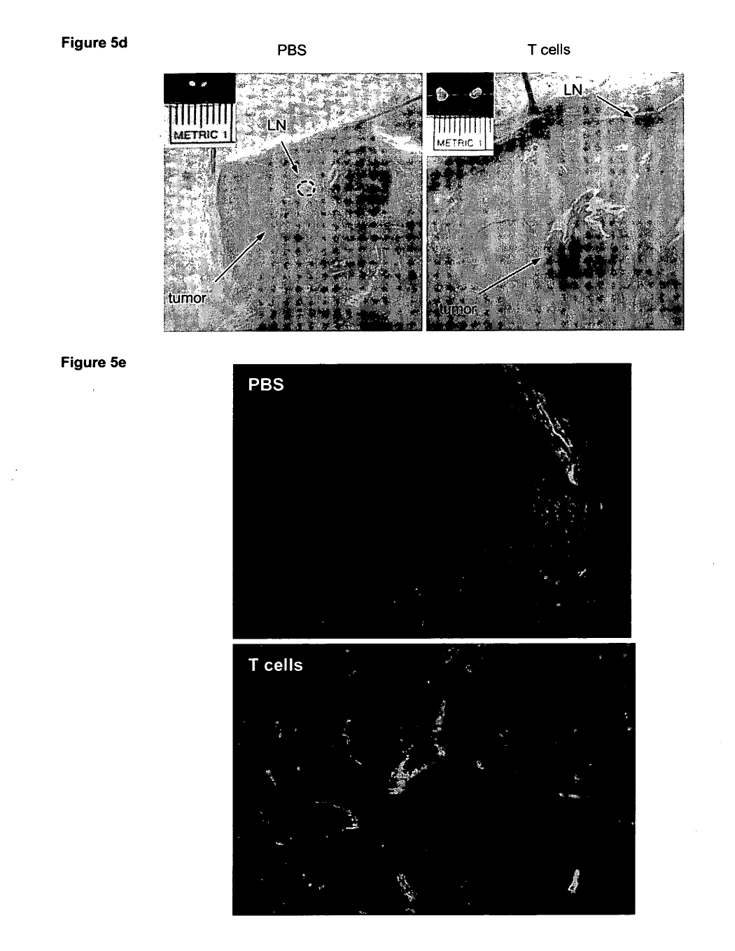

Reconstitution of Hs578T breast cancer tumors with T cells isolated from the donor PBMC (both CD4⁺ and CD8⁺) resulted in accelerated tumor development (FIG. 5a). CD4⁺T cells also promoted the development of MCF-7 breast cancer tumors (FIG. 5a) but not that of 1806 breast cancer cells (not shown). This acceleration of tumor development was reproducible in cohorts of mice generated with human cells from different donors (FIG. 5b). Furthermore, it was dependent on CD4⁺T cells as reconstitution with purified CD8⁺T cells had not had any impact on the early tumor development (FIGS. 5a and b).

Next, whether previously primed CD4⁺T cells can confer the acceleration of tumor development was analyzed. Humanized mice were constructed, Hs578T breast cancer tumors implanted and then injected with CD4⁺T cells isolated from the donor PBMC. At day 15, at the peak of breast tumor development, tumors were harvested, pooled from several mice and CD4⁺T cells were sorted. These in vivo primed CD4⁺T cells were then injected into “T cell naïve” Hs578T breast cancer tumors of recipient humanized mice constructed with CD34⁺HPCs from the same donor. Control mice received PBS. A single transfer of 1.5×10⁶ in vivo primed CD4⁺T cells per mouse led to acceleration of breast cancer tumor development in three out of four tested mice in two independent experiments (mean tumor volume±SEM=51±17 in control mice that received PBS vs 167±27 in experimental mice that received T cells, p=0.06; FIG. 5c). Thus, human breast cancer tumors develop faster in the presence of human CD4⁺T cells.

Increased tumor volume was associated with angiogenesis. Macroscopic analysis of Hs578T breast cancer tumors injected with PBS demonstrated at day 15 a clearly visible tumor and a small draining lymph node (FIG. 5d). Strikingly, when tumors were injected with CD4⁺T cells both the tumor and the draining lymph node were enlarged with visible blood vessels (FIG. 5d). Enhanced vascularization was demonstrated by administering FITC-lectin into mice whose tumors have been injected with CD4⁺T cells (FIG. 5e).

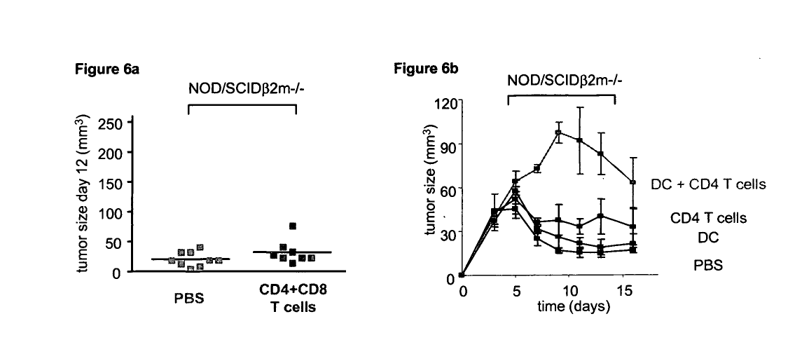

CD4⁺T cells require DCs to promote breast cancer tumor development. To determine whether CD4⁺T cells acted directly on breast cancer cells the effect of CD4+ cells in NOD/SCID/β2m⁻ mice bearing breast cancer tumor in the absence of human immune cells (no CD34⁺HPCs transplant) was assessed. As shown in FIG. 6a, no change in tumor volume was observed upon injection of T cells isolated from different donors. This suggested that the pro-cancer effect of CD4⁺T cells required a cell generated from CD34⁺HPCs transplant, possibly DCs. Therefore, DCs were generated by culturing monocytes with GM-CSF and IL-4 and injected them together with autologous CD4⁺T cells in mice bearing Hs578T breast cancer tumors. As shown in FIG. 6b, the injection of either DCs or T cells did not result in the change in tumor volume. However, co-injection of DCs and T cells led to acceleration of breast cancer tumors development (FIG. 6b). Thus, DCs are necessary for CD4⁺T cells to promote early development of breast cancer tumors.

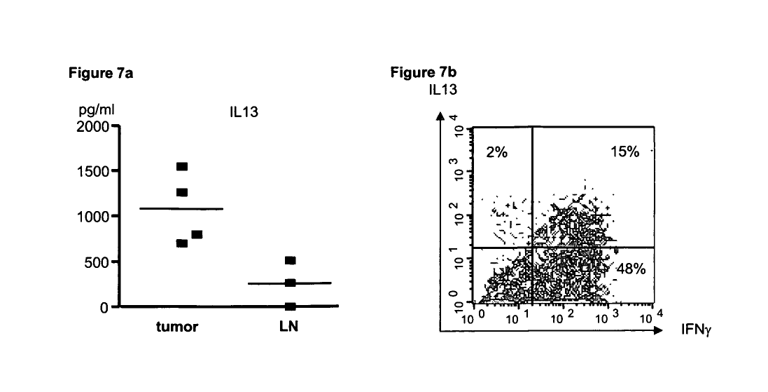

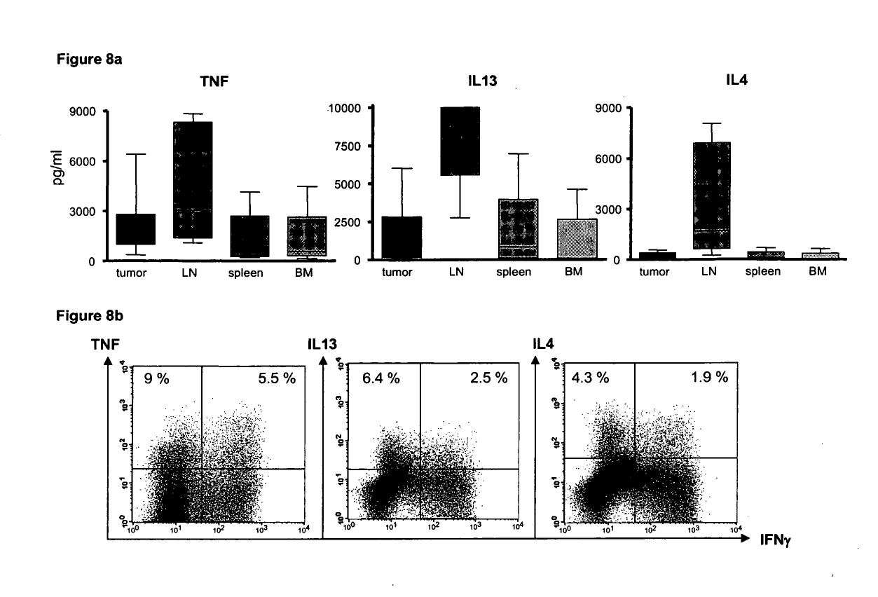

CD4⁺T cells are polarized to secrete IL-13. Next, CD4⁺T cells were isolated from breast cancer tumors in humanized mice. It was found that the CD4⁺T cells were isolated from breast cancer tumors were polarized towards secretion of type 2 cytokines. CD4⁺T cells were sorted from tumors and their draining lymph nodes at day 15, activated with PMA and lonomycin and cytokines were assessed in the supernatants. CD4⁺T cells secreted large amounts (>10 ng/ml) of IL-2 and IFN-γ but also IL-4, IL-13 and TNF (FIG. 8a and not shown). FIG. 7a shows high levels of IL-13 (1080±200 pg/ml) that could be detected, particularly in CD4⁺T cells infiltrating tumors. Flow cytometry demonstrated intracytoplasmic expression of IL-13 in up to 17% of CD4⁺T cells (13%±3% IL-13⁺CD4⁺T cells; FIG. 7b). Most of IL-13 expressing CD4⁺T cells also expressed IFN-γ (FIG. 7b) resembling the pattern of expression found in some of the patient tumors.

DCs infiltrating breast cancer tumors polarize CD4⁺T cells to secrete type 2 cytokines. To further investigate the influence of tumor associated DCs on CD4⁺T cell function, human DCs from NOD/SCID/β2m⁻¹ mice transplanted with CD34⁺HPCs and implanted with Hs578T breast cancer tumors were studied. DCs were isolated from breast cancer tumors, their draining lymph nodes, spleen and bone marrow 4 days after tumor implantation and tested for their ability to polarize naive allogeneic CD4⁺T cells in vitro. After 5 days, CD4⁺T cells were activated with PMA and lonomycin and cytokines were assessed in the supernatants. DCs isolated from all analyzed tissues induced allogeneic CD4⁺T cells to secrete large amounts of IL-2 and IFN-γ (>10 ng/ml, not shown). Furthermore, DCs isolated from both breast cancer tumors and particularly from their draining lymph nodes primed CD4⁺T cells to secrete high levels of TNF, IL-13, and IL-4 (mean concentration±SEM=4491±1599 pg/ml TNF; 7961±1342 pg/ml IL-13 and 3345±1508 pg/ml IL-4; FIG. 8a). This pattern of cytokine secretion was sustained over time and DC sorted from day 30 tumor triggered even higher secretion of type 2 and pro-inflammatory cytokines (not shown). Flow cytometry analysis of CD4⁺T cells primed by DCs isolated from day 4 tumors showed approximately 9% of CD4⁺T cells expressing IL-13, 6% expressing IL-4 and 15% expressing TNF (FIG. 8b). The pattern of staining was consistent with Th2 polarization, and the majority of CD4⁺T cells expressing IL-13 and/or IL-4 was single positive and did not express IFN-γ (FIG. 8b). Thus, breast cancer polarizes DCs to prime a fraction of CD4⁺T cells to produce type 2 (IL-4 and IL-13) and proinflammatory (TNF, IFN-γ) cytokines. Comparable numbers of IL-13 expressing CD4⁺T cells were detected in cultures with total naive CD4⁺T cells or with naive CD4⁺T cells depleted of Vα24⁺ cells. Thus, DCs are imprinted by breast cancer to polarize CD4⁺T cells towards secretion of type cytokines.

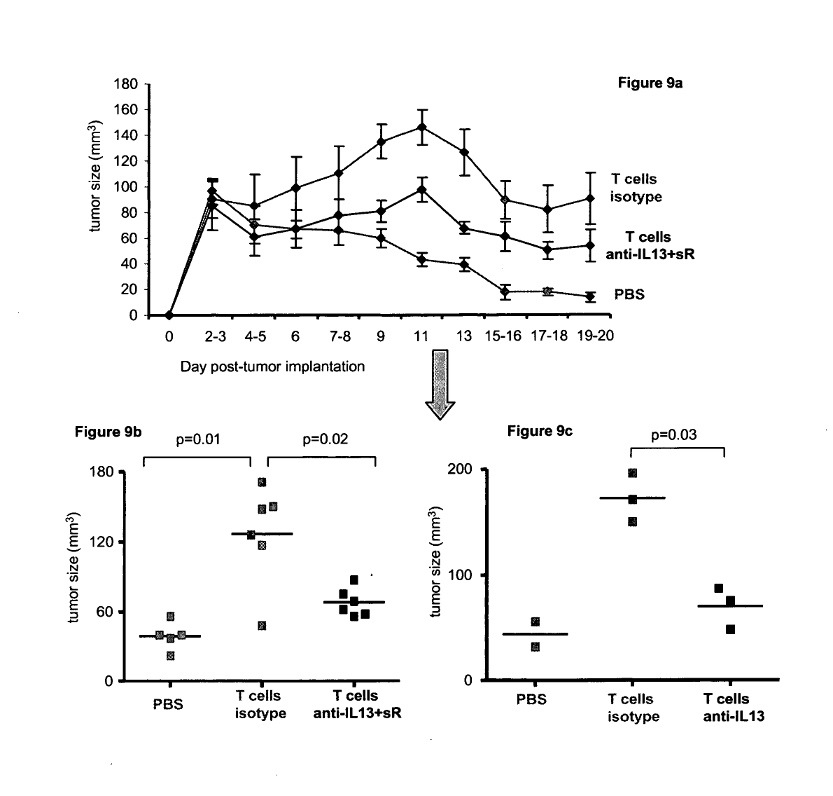

CD4⁺T cells promote tumor development via IL-13. It was found that human breast cancer tumors developed faster in the microenvironment of human CD4⁺T cells and skewed them to secrete IL-13. This together with earlier reports on an immunoregulatory role of IL-13 in cancer (Terabe et al., 2000) suggested that IL-13 might be involved. To establish this, humanized mice bearing Hs587T breast cancer tumors were treated with IL-13 antagonists, an antibody neutralizing IL-13 and a soluble IL-13R. It was found that mice reconstituted with T cells, and treated with isotype control, showed accelerated tumor development throughout three weeks follow up (FIG. 9a). Meanwhile, mice treated with IL-13 antagonists showed sustained inhibition of tumor development (FIG. 9a). Tumor volume at day 13 was 39±5 mm in animals without T cells (PBS control) and 128±18 in animals with T cells (mean tumor volume±SEM=p=0.01; FIG. 9b). Mice treated With IL-13 antagonists showed significant inhibition of tumor development (mean tumor volume at day 13±SEM=128±18 in animals treated with isotype control and 68±5 in animals treated with IL-13 antagonists; p=0.02; FIG. 9b). Finally, treatment with neutralizing IL-13 mAb alone was sufficient to prevent acceleration in breast cancer tumor development (mean tumor volume at day 11=172±13 in animals treated with isotype control and 70±11.5 in animals treated with IL-13 neutralizing mAb; p=0.03; FIG. 9c). Thus, accelerated development of breast cancer tumors in Humouse reconstituted with CD4⁺T cells can be counteracted by treatment with IL-13 antagonists.

The presence of mature DCs outside lymphoid organ is associated with inflammation either septic as for example in infections or aseptic as for example in autoimmune diseases (Blanco et al., 2001; Radstake et al., 2005; Thomas et al., 1999). The present inventors had previsouly found infiltration of breast cancer tumors with mature DCs in patients (Bell et al., 1999). The immunological consequences of the presence of DCs in breast cancer tumor microenvironment was analyzed. It was found that CD4⁺T cells secreting type 1 and type 2 cytokines (predominantly IL-13) in tumor samples from patients with breast cancer. Because limited amount of tumor material that can be obtained from patients hampers causative studies, NOD/SCID β2m^{-/-} mice engrafted with human CD34⁺ hematopoietic progenitor cells (HPCs) and implanted with human breast cancer cell lines were used to demonstrate the link between breast cancer, DCs and CD4⁺T cell polarization. Therefore, it is demonstrated herein that breast cancer cells attract human DCs and imprint them to polarize naïve CD4⁺T cells to secrete type 2 cytokines including IL-13. CD4⁺T cells promote early tumor development, and this is associated with enhanced angiogenesis. This pro-cancer effect can be prevented with IL-13 antagonists.

To established whether IL-13 is autocrine, as is the case in Hodgkin's disease (Kapp et al., 1999), or paracrine secreted by CD4⁺T cells and perhaps accessory cells such as mast cells, the source(s) of the IL-13 were explored. The source of IL-13 could influence the mechanism through which it would regulate tumor development in vivo (Fichtner-Feigl et al., 2006). For example, IL-13 could have a direct effect on breast cancer cells. Surprisingly, it was found that breast cancer cells in tumors from patients express IL-13. Earlier in vitro studies using breast cancer cell lines and recombinant IL-13 (as well as IL-4) demonstrated inhibition of estrogen-induced proliferation and acquisition of breast cancer marker, gross cystic disease fluid protein-15 (GCDFP-15) (Blais et al., 1996; Serve et al., 1996). IL-13 has also been indicated in the control of sex steroid biosynthesis from adrenal precursors (Gingras et al., 1999). In line with possible direct effect of IL-13 in vivo are recent findings on the expression of IL13Rα2 in highly aggressive variants of breast cancer with propensity to from lung metastasis (Minn et al., 2005). An indirect pathway may involve IL-13-mediated polarization of tumor infiltrating macrophages towards M2 cells (Sinha et al., 2005). These in turn promote angiogenesis (Mantovani et al., 2005) and/or secrete factors inhibiting anti-cancer effector function of CD8⁺T cells, for example TGF-β (Ghiringhelli et al., 2005; Li et al., 2005; Terabe et al., 2003) thereby amplifying tumor development.

Two patterns of cytokine expression by CD4⁺T cells were observed. One, with the presence of single positive CD4⁺T cells expressing either IL-13 or IFN-γ, consistent with the classical definition of type 2 polarization (Mosmann and Coffman, 1989). The pattern of expression suggests the bona fide Th1 and Th2 cells in breast cancer tumor microenvironment. The second pattern showed actually two subsets of IL-13-expressing CD4⁺T cells: single positive (bone fide Th2) and double positive IL-13 and IFN-γ. The latter pattern is reminiscent of IL-4 and IFN-γ expression observed in adult NKT cells (Kadowaki et al., 2001). These results raise a possibility that breast cancer tumors are infiltrated with NKT cells. However, since depletion of Vα24-expressing cells did not abolish induction of IL-13 in the vitro studies, a possible role of non-classical Vα24-negative NKT cells must be considered. Nevertheless, these results demonstrate the presence of two types of CD4⁺T cells secreting type 2 cytokines in breast cancer microenvironment.

The presence of type 2 cytokines together with TNF producing CD4⁺T cells resembles the pro-inflammatory type 2 responses induced by TSLP-primed DCs (Soumelis et al., 2002) and mediated via OX40 ligand (Ito et al., 2005). These molecules may be present in the microenvironment of breast cancer. An alternative pathway could include MUC-1 whose potential role in the attraction of myeloid DCs and Th2 polarization has been suggested in the in vitro studies (Carlos et al., 2005). Nevertheless, the priming of these CD4⁺T cells in vivo is dependent on antigen presentation by autologous DCs as transfer of CD4⁺T cells only did not promote tumor development in humanized mice. Other studies by the present inventors suggest that the effector phase, i.e., accelerated tumor development upon transfer of in vivo primed CD4⁺T cells to naive mice, also depends on the presence of tumor infiltrating DCs autologous to T cells.

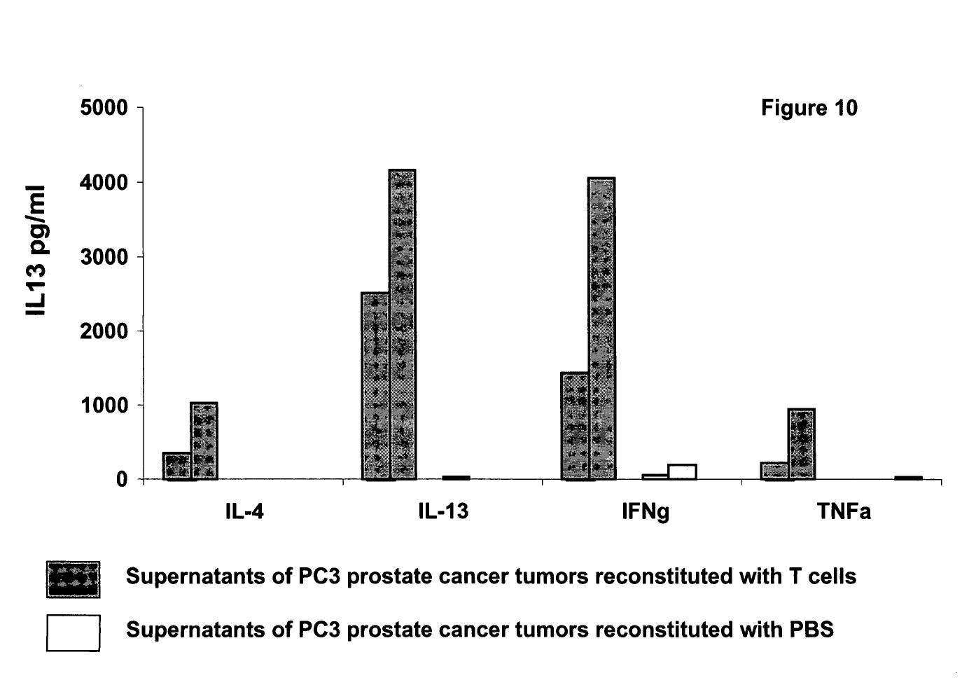

Prostate cancer tumors were established using PC3 cell line and reconstituted at days 3 and 6 with T cells (a mixture of CD4+ and CD8+T cells). Control mice received PBS at day 20 post-T cell transfer, tumors were harvested and put in overnight culture with PMA and lonomycin. As shown in FIG. 10, tumors reconstituted with CD4⁺T cells but not control, showed high levels of IL-13 in supernatants. These results demonstrate that the observations from breast cancer tumors can be extended to prostate cancer, another tumor of epithelial origin.

In conclusion, combining the studies of human cancer using ex vivo analysis of patient samples and in vivo analysis in the model of the human immune system and human cancer it is demonstrated herein that breast cancer attracts human DCs and imprints them to prime CD4⁺T cells into pro-cancer type 2 immunity. This can be prevented with IL-13 antagonists as target for therapy in cancers that depend on IL-13 production, e.g., breast cancer.

The model can be used at both basic and clinical level. At the basic level it will permit to determine the mechanisms tumors use to escape the immune system and to identify molecules the targeting of which might be used for therapy. At the clinical level the OncoHumouse will eventually permit us to design strategies to eliminate tumor cells through the manipulation of the immune system such as vaccination, antibody therapy, and adoptive transfer coupled or not to traditional chemotherapy regimens.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Anderson, T. M., Hess, S. D., Egilmez, N. K., Nwogu, C. E., Lenox, J. M., and Bankert, R. B. (2003). Comparison of human lung cancer/SCID mouse tumor xenografts and cell culture growth with patient clinical outcomes. J Cancer Res Clin Oncol 129, 565-568.
Balkwill, F., Charles, K. A., and Mantovani, A. (2005). Smoldering and polarized inflammation in the initiation and promotion of malignant disease. Cancer Cell 7, 211-217.
Banchereau, J., Briere, F., Caux, C., Davoust, J., Lebecque, S., Liu, Y., Pulendran, B., and Palucka, K. (2000). Immunobiology of dendritic cells. Ann Rev Immunol 18, 767-811.
Banchereau, J., and Palucka, A. K. (2005). Dendritic cells as therapeutic vaccines against cancer. Nat Rev Immunol 5, 296-306.
Banchereau, J., Pascual, V., and Palucka, A. K. (2004). Autoimmunity through Cytokine-Induced Dendritic Cell Activation. Immunity 20, 539-550.
Banchereau, J., and Steinman, R. M. (1998). Dendritic cells and the control of immunity. Nature 392, 245-252.
Bankert, R. B., Egilmez, N. K., and Hess, S. D. (2001). Human-SCID mouse chimeric models for the evaluation of anti-cancer therapies. Trends Immunol 22, 386-393.
Bell, D., Chomarat, P., Broyles, D., Netto, G., Harb, G. M., Lebecque, S., Valladeau, J., Davoust, J., Palucka, K. A., and Banchereau, J. (1999). In breast carcinoma tissue, immature dendritic cells reside within the tumor, whereas mature dendritic cells are located in peritumoral areas. J Exp Med 190, 1417-1426.
Blais, Y., Gingras, S., Haagensen, D. E., Labrie, F., and Simard, J. (1996). Interleukin-4 and interleukin-13 inhibit estrogen-induced breast cancer cell proliferation and stimulate GCDFP-15 expression in human breast cancer cells. Mol Cell Endocrinol 121, 11-18.
Blanco, P., Palucka, A. K., Gill, M., Pascual, V., and Banchereau, J. (2001). Induction of dendritic cell differentiation by IFN-alpha in systemic lupus erythematosus. Science 294, 1540-1543.
Carlos, C. A., Dong, H. F., Howard, O. M., Oppenheim, J. J., Hanisch, F. G., and Finn, O. J. (2005). Human tumor antigen MUC1 is chemotactic for immature dendritic cells and elicits maturation but does not promote Th1 type immunity. J Immunol 175, 1628-1635.
Chomarat, P., Banchereau, J., Davoust, J., and Palucka, A. K. (2000). IL-6 switches the differentiation of monocytes from dendritic cells to macrophages. Nat Immunol 1, 510-514.
Condeelis, J., and Pollard, J. W. (2006). Macrophages: obligate partners for tumor cell migration, invasion, and metastasis. Cell 124, 263-266.
Cosmi, L., Annunziato, F., Galli, M. I. G., Maggi, R. M. E., Nagata, K., and Romagnani, S. (2000). CRTH2 is the most reliable marker for the detection of circulating human type 2 Th and type 2 T cytotoxic cells in health and disease. Eur J Immunol 30, 2972-2979.
Coussens, L. M., and Werb, Z. (2002). Inflammation and cancer. Nature 420, 860-867.
Delamarre, L., Pack, M., Chang, H., Mellman, I., and Trombetta, E. S. (2005). Differential lysosomal proteolysis in antigen-presenting cells determines antigen fate. Science 307, 1630-1634.
Enk, A. H., Jonuleit, H., Saloga, J., and Knop, J. (1997). Dendritic cells as mediators of tumor-induced tolerance in metastatic melanoma. Int J Cancer 73, 309-316.
Fichtner-Feigl, S., Strober, W., Kawakami, K., Puri, R. K., and Kitani, A. (2006). IL-13 signalling through the IL-13alpha2 receptor is involved in induction of TGF-beta1 production and fibrosis. Nat Med 12, 99-106.
Gabrilovich, D. (2004). Mechanisms and functional significance of tumour-induced dendritic-cell defects. Nat Rev Immunol 4, 941-952.
Gabrilovich, D. I., Chen, H. L., Girgis, K. R., Cunningham, H. T., Meny, G. M., Nadaf, S., Kavanaugh, D., and Carbone, D. P. (1996). Production of vascular endothelial growth factor by human tumors inhibits the functional maturation of dendritic cells. Nat Med 2, 1096-1103.
Ghiringhelli, F., Puig, P. E., Roux, S., Parcellier, A., Schmitt, E., Solary, E., Kroemer, G., Martin, F., Chauffert, B., and Zitvogel, L. (2005). Tumor cells convert immature myeloid dendritic cells into TGF-beta-secreting cells inducing CD4+CD25+ regulatory T cell proliferation. J Exp Med 202, 919-929.
Gingras, S., Moriggl, R., Groner, B., and Simard, J. (1999). Induction of 3beta-hydroxysteroid dehydrogenase/delta5-delta4 isomerase type 1 gene transcription in human breast cancer cell lines and in normal mammary epithelial cells by interleukin-4 and interleukin-13. Mol Endocrinol 13, 66-81.
Gordon, S., and Taylor, P. R. (2005). Monocyte and macrophage heterogeneity. Nat Rev Immunol 5, 953-964.
Ito, T., Wang, Y. H., Duramad, O., Hori, T., Delespesse, G. J., Watanabe, N., Qin, F. X., Yao, Z., Cao, W., and Liu, Y. J. (2005). TSLP-activated dendritic cells induce an inflammatory T helper type 2 cell response through OX40 ligand. J Exp Med 202, 1213-1223.
Iwanuma, Y., Chen, F. A., Egilmez, N. K., Takita, H., and Bankert, R. B. (1997). Antitumor immune response of human peripheral blood lymphocytes coengrafted with tumor into severe combined immunodeficient mice. Cancer Res 57, 2937-2942.
Janeway, C. A., Jr., and Medzhitov, R. (2002). Innate immune recognition. Annu Rev Immunol 20, 197-216.
Joyce, J. A. (2005). Therapeutic targeting of the tumor microenvironment. Cancer Cell 7, 513-520.
Kadowaki, N., Antonenko, S., Ho, S., Rissoan, M. C., Soumelis, V., Porcelli, S. A., Lanier, L. L., and Liu, Y. J. (2001). Distinct cytokine profiles of neonatal natural killer T cells after expansion with subsets of dendritic cells. J Exp Med 193, 1221-1226.
Kamel-Reid, S., Letarte, M., Doedens, M., Greaves, A., Murdoch, B., Grunberger, T., Lapidot, T., Thomer, P., Freedman, M. H., Phillips, R. A., and et al. (1991). Bone marrow from children in relapse with pre-B acute lymphoblastic leukemia proliferates and disseminates rapidly in scid mice. Blood 78, 2973-2981.
Kamel-Reid, S., Letarte, M., Sirard, C., Doedens, M., Grunberger, T., Fulop, G., Freedman, M. H., Phillips, R. A., and Dick, J. E. (1989). A model of human acute lymphoblastic leukemia in immune-deficient SCID mice. Science 246, 1597-1600.
Kapp, U., Yeh, W. C., Patterson, B., Elia, A. J., Kagi, D., Ho, A., Hessel, A., Tipsword, M., Williams, A., Mirtsos, C., et al. (1999). Interleukin 13 is secreted by and stimulates the growth of Hodgkin and Reed-Sternberg cells. J Exp Med 189, 1939-1946.
Kelland, L. R. (2004). Of mice and men: values and liabilities of the athymic nude mouse model in anticancer drug development. Eur J Cancer 40, 827-836.
Levings, M. K., Gregori, S., Tresoldi, E., Cazzaniga, S., Bonini, C., and Roncarolo, M. G. (2005). Differentiation of Tr1 cells by immature dendritic cells requires IL-10 but not CD25+CD4+ Tr cells. Blood 105, 1162-1169.
Li, M. O., Wan, Y. Y., Sanjabi, S., Robertson, A. K., and Flavell, R. A. (2005). Transforming Growth Factor-beta Regulation of Immune Responses. Annu Rev Immunol.
Mantovani, A., Sica, A., and Locati, M. (2005). Macrophage polarization comes of age. Immunity 23, 344-346.
Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.
Mosier, D. E., Gulizia, R. J., Baird, S. M., and Wilson, D. B. (1988). Transfer of a functional human immune system to mice with severe combined immunodeficiency. Nature 335, 256-259.
Mosmann, T. R., and Coffman, R. L. (1989). TH1 and TH2 cells: different patterns of lymphokine secretion lead to different functional properties. Annu Rev Immunol 7, 145-173.
Mueller, B. M., and Reisfeld, R. A. (1991). Potential of the scid mouse as a host for human tumors. Cancer Metastasis Rev 10, 193-200.
Muller, A., Homey, B., Soto, H., Ge, N., Catron, D., Buchanan, M. E., McClanahan, T., Murphy, E., Yuan, W., Wagner, S. N., et al. (2001). Involvement of chemokine receptors in breast cancer metastasis. Nature 410, 50-56.
Nagata, K., Tanaka, K., Ogawa, K., Kemmotsu, K., Imai, T., Yoshie, O., Abe, H., Tada, K., Nakamura, M., Sugamura, K., and Takano, S. (1999). Selective expression of a novel surface molecule by human Th2 cells in vivo. J Immunol 162, 1278-1286.
Palucka, A. K., Gatlin, J., Blanck, J. P., Melkus, M. W., Clayton, S., Ueno, H., Kraus, E. T., Cravens, P., Bennett, L., Padgett-Thomas, A., et al. (2003). Human dendritic cell subsets in NOD/SCID mice engrafted with CD34+ hematopoietic progenitors. Blood 102, 3302-3310.
Palucka, A. K., Scuderi, R., Porwit, A., Jeha, S., Gruber, A., Bjorkholm, M., Beran, M., and Pisa, P. (1996). Acute lymphoblastic leukemias from relapse engraft more rapidly in SCID mice. Leukemia 10, 558-563.
Pulendran, B., Banchereau, J., Burkeholder, S., Kraus, E., Guinet, E., Chalouni, C., Caron, D., Maliszewski, C., Davoust, J., Fay, J., and Palucka, K. (2000). Flt3-ligand and granulocyte colony-stimulating factor mobilize distinct human dendritic cell subsets in vivo. J Immunol 165, 566-572.
Radstake, T. R., van Lieshout, A. W., van Riel, P. L., van den Berg, W. B., and Adema, G. J. (2005). Dendritic cells, Fc{gamma} receptors, and Toll-like receptors: potential allies in the battle against rheumatoid arthritis. Ann Rheum Dis 64, 1532-1538.
Reddy, S., Piccione, D., Takita, H., and Bankert, R. B. (1987). Human lung tumor growth established in the lung and subcutaneous tissue of mice with severe combined immunodeficiency. Cancer Res 47, 2456-2460.
Sakakibara, T., Xu, Y., Bumpers, H. L., Chen, F. A., Bankert, R. B., Arredondo, M. A., Edge, S. B., and Repasky, E. A. (1996). Growth and Metastasis of Surgical Specimens of Human Breast Carcinomas in SCID Mice. Cancer J Sci Am 2, 291.
Serve, H., Oelmann, E., Herweg, A., Oberberg, D., Serve, S., Reufi, B., Mucke, C., Minty, A., Thiel, E., and Berdel, W. E. (1996). Inhibition of proliferation and clonal growth of human breast cancer cells by interleukin 13. Cancer Res 56, 3583-3588.
Shortman, K., and Liu, Y. J. (2002). Mouse and human dendritic cell subtypes. Nature Rev Immunol 2, 151-161.
Sinha, P., Clements, V. K., and Ostrand-Rosenberg, S. (2005). Interleukin-13-regulated M2 macrophages in combination with myeloid suppressor cells block immune surveillance against metastasis. Cancer Res 65, 11743-11751.
Soumelis, V., Reche, P. A., Kanzler, H., Yuan, W., Edward, G., Homey, B., Gilliet, M., Ho, S., Antonenko, S., Lauerma, A., et al. (2002). Human epithelial cells trigger dendritic cell mediated allergic inflammation by producing TSLP. Nat Immunol 3, 673-680.
Steinman, R. M. (1991). The dendritic cell system and its role in immunogenicity. Annu Rev Immunol 9, 271-296.
Terabe, M., Matsui, S., Noben-Trauth, N., Chen, H., Watson, C., Donaldson, D. D., Carbone, D. P., Paul, W. E., and Berzofsky, J. A. (2000). NKT cell-mediated repression of tumor immunosurveillance by IL-13 and the IL-4R-STAT6 pathway. Nat Immunol 1, 515-520.
Terabe, M., Matsui, S., Park, J. M., Mamura, M., Noben-Trauth, N., Donaldson, D. D., Chen, W., Wahl, S. M., Ledbetter, S., Pratt, B., et al. (2003). Transforming growth factor-beta production and myeloid cells are an effector mechanism through which CD1d-restricted T cells block cytotoxic T lymphocyte-mediated tumor immunosurveillance: abrogation prevents tumor recurrence. J Exp Med 198, 1741-1752.
Thomas, R., MacDonald, K. P., Pettit, A. R., Cavanagh, L. L., Padmanabha, J., and Zehntner, S. (1999). Dendritic cells and the pathogenesis of rheumatoid arthritis. J Leukoc Biol 66, 286-292.
Uckun, F. M., Sather, H., Reaman, G., Shuster, J., Land, V., Trigg, M., Gunther, R., Chelstrom, L., Bleyer, A., Gaynon, P., and et al. (1995). Leukemic cell growth in SCID mice as a predictor of relapse in high-risk B-lineage acute lymphoblastic leukemia. Blood 85, 873-878.
Wang, T., Niu, G., Kortylewski, M., Burdelya, L., Shain, K., Zhang, S., Bhattacharya, R., Gabrilovich, D., Heller, R., Coppola, D., et al. (2004). Regulation of the innate and adaptive immune responses by Stat-3 signaling in tumor cells. Nat Med 10, 48-54.

What is claimed is:

1. A method of improving a T cell response to cancer comprising:
identifying a patient in need of cancer treatment in which the predominate immune response includes the secretion of Type II cytokines; and
treating the affected tissue with one or more Type II cytokine antagonists, wherein the Type II cytokine antagonists block CD4+ T cells that secrete Type II cytokines and increase the percentage of Th1 T cells in the affected tissue.

2. The method of claim 1, wherein the Type II cytokine antagonists comprises anti-IL-4, IL-5, IL-9, IL-13, or IL-25 antibody, and combinations thereof; a humanized anti-IL-4, IL-5, IL-9, IL-13, or IL-25 antibody, and combinations thereof; inactivated IL-4, IL-5, IL-9, IL-13 or IL-25 and combinations thereof; soluble IL-4, IL-5, IL-9, IL-13 or IL-25 receptors and combinations thereof.

3. The method of claim 1, wherein the cancer comprises cancers of epithelial origin.

4. The method of claim 1, wherein the cancer comprises breast cancer or prostate cancer.

5. The method of claim 1, wherein the Type II cytokine antagonist comprises a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.

6. The method of claim 1, wherein the IL-13 antagonist decreases CD4+ T cells that secrete Type II cytokines and increases the percentage of CD4+ T cells that secrete Type I cytokines.

7. The method of claim 1, wherein the antagonists further comprise an anti-IFN-γ antibody, a humanized anti-IFN-γ antibody, a soluble IFN-γ receptor and combinations thereof.

8. The method of claim 1, wherein the antagonists further comprise an anti-TNF antibody, a humanized anti-TNF antibody, a soluble TNF receptor and combinations thereof.

9. A method of improving T cell responses to breast cancer comprising:
identifying a patient in need of treatment for a breast cancer; and
treating the affected tissue with one or more IL-13 antagonists, wherein the IL-13 antagonists block CD4+ T cells that secrete Type II cytokines.

10. The method of claim 9, wherein the IL-13 antagonist comprises an anti-IL-13 antibody, a humanized anti-IL-13 antibody.

11. The method of claim 9, wherein the IL-13 antagonist comprises an antagonist of IL-13-IL-13 receptor binding.

12. The method of claim 9, wherein the IL-13 antagonist comprises a blocking a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.

13. The method of claim 9, wherein the IL-13 antagonist decreases CD4+ T cells that secrete Type II cytokines and increases CD4+ T cells that secrete Type I cytokines.

14. The method of claim 9, wherein the antagonists further comprise an anti-IFN-γ antibody, a humanized anti-IFN-γ antibody and combinations thereof.

15. The method of claim 9, wherein the antagonists further comprise an anti-IFN-γ antibody, a humanized anti-IFN-γ antibody, a soluble IFN-Y receptor and combinations thereof.

16. The method of claim 9, wherein the antagonists further comprise an anti-TNF antibody, a humanized anti-TNF antibody, a soluble TNF receptor and combinations thereof.

17. A composition that improves immunity against breast cancer comprising:
a therapeutically effective amount of one or more Type II cytokine antagonists.

18. The composition of claim 17, wherein the Type II cytokine antagonists comprises anti-IL-4, IL-5, IL-9, IL-13, or IL-25 antibody, a humanized anti-IL-4, IL-5, IL-9, IL-13, IL-25 antibody, and combinations thereof.

19. The composition of claim 17, wherein the Type II cytokine antagonists comprises inactivated IL-4, IL-5, IL-9, IL-13 or IL-25 and combinations thereof

20. The composition of claim 17, wherein the Type II cytokine antagonists comprises soluble IL-4, IL-5, IL-9, IL-13 or IL-25 receptors and combinations thereof.

21. The composition of claim 17, wherein the antagonists further comprise an anti-IFN-γ antibody, a humanized anti-IFN-γ antibody, a soluble IFN-γ receptor and combinations thereof.

22. The composition of claim 17, wherein the antagonists further comprise an anti-TNF antibody, a humanized anti-TNF antibody, a soluble TNF receptor and combinations thereof.

23. The composition of claim 17, further comprising one or more Type I cytokines that stimulate Th1 responses.

24. The composition of claim 17, further comprising one or more Type I cytokines in a single dose that stimulate Th1 responses.

25. A method of reducing Th2 polarization by human breast cancer comprising:
providing an effective amount of one or more Type II cytokine antagonists selected from anti-IL-4, IL-5, IL-9, IL-13 or IL-25; soluble receptors for IL-4, IL-5, IL-9, IL-13 or IL-25 and combinations thereof.

26. The method of claim 25, further comprising providing the patient with an anti-IFN-γ antibody, a humanized anti-IFN-γ antibody, a soluble IFN-γ receptor and combinations thereof.

27. The method of claim 25, further comprising providing the patient with an anti-TNF antibody, a humanized anti-TNF antibody, a soluble TNF receptor and combinations thereof.

28. The method of claim 25, wherein Type II cytokine antagonist comprises an IL-13 selected from a blocking IL-13 receptor binding antibody, an inactivated, IL-13, a soluble IL-13R and combinations thereof.

29. A method for inhibiting angiogenesis in tumors comprising:
providing an effective amount of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.

30. A method for inhibiting the function of tumor associated macrophages by cancers of epithelial cell origin comprising:
providing an effective amount of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.

31. A method for prevention of metastasis comprising:
providing an amount effective to prevent metastasis of cancer of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.

32. A method for prevention of the formation of tumor stroma comprising:
providing an amount effective to prevention of the formation of tumor stroma of one or more IL-13 antagonists selected from a blocking anti-IL-13 cytokine receptor, anti-IL-13 neutralizing antibodies, anti-IL-13 receptor antagonists, anti-IL-13 soluble receptors, molecules that interfere with the anti-IL-13 receptor-ligand binding, inhibitors of downstream events of anti-IL-13, an inactivated IL-13 and combinations thereof.
